SEQUENCE LISTING (15 sequences)

SEQ ID NO: 1 - protein, 15 aa, Homo sapiens
EPKSCDKTHTCPPCP

SEQ ID NO: 2 - protein, 12 aa, Homo sapiens
ERKCCVECPPCP

SEQ ID NO: 3 - protein, 32 aa, Homo sapiens
ELKTPLGDTTHTCPRCPEPKSCDTPPPCPRCP

SEQ ID NO: 4 - protein, 12 aa, Homo sapiens
ESKYGPPCPSCP

SEQ ID NO: 5 - protein, 232 aa, Homo sapiens
  misc_feature (141)..(141): Xaa can be any naturally occurring amino acid
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPGK

SEQ ID NO: 6 - protein, 228 aa, Homo sapiens
  misc_feature (63)..(63): Xaa can be any naturally occurring amino acid
ERKCCVECPPCPAPPVAGPSVFLFPPKPKDTLMISRTPEVTCVVVDVSHEDPEVQFNWYVDGXEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIXVEWESNGQPENNYKTTPPMLDSDGSFFLYSKLTVDKSRWQQGNVFSCSVMHEALHNHYTQKSLSLSPGK

SEQ ID NO: 7 - protein, 279 aa, Homo sapiens
  misc_feature (123)..(124): Xaa can be any naturally occurring amino acid
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKXXEEQXNSTFRVVSVLTVXHQDWLNGKEYKCKVSNKALPAPIEKTISKXKGQPREPQVYTLPPSREEMTKNQVSLTCLVKGFYPSDIAXEWESXGQPENXYNTTPPXLDSDGSFFLYSXLTVDKSRWQXGNXFSCSVMHEALHNXXTQKSLSLSPGK

SEQ ID NO: 8 - protein, 229 aa, Homo sapiens
  misc_feature (191)..(191): Xaa can be any naturally occurring amino acid
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSXLTVDKSRWQEGNVFSCSVMHEALHNHYTQKSLSLSLGK

SEQ ID NO: 9 - protein, 211 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 211 aa, Homo sapiens
GPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 211 aa, Homo sapiens
GPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 12 - protein, 211 aa, Homo sapiens
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 210 aa, Mus musculus
SSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 14 - protein, 211 aa, Mus musculus
GPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 211 aa, Mus musculus
GPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

(19) United States
(12) Patent Application Publication
(10) Pub. No.: US 2013/0177555 A1
(43) Pub. Date: Jul. 11, 2013

(54) Monomeric Polypeptides Comprising Variant FC Regions And Methods Of Use

(71) Applicants: Ian Wilkinson, Bedford (GB); Carl Innes Webster, Cambridge (GB); David Christopher Lowe, Cambridge (GB)

(72) Inventors: Ian Wilkinson, Bedford (GB); Carl Innes Webster, Cambridge (GB); David Christopher Lowe, Cambridge (GB)

(73) Assignee: MEDIMMUNE LIMITED, Cambridge (UK)

(21) Appl. No.: 13/814,657

(22) PCT Filed: Aug. 11, 2011
(86) PCT No.: PCT/EP2011/063857

§ 371 Date: Mar. 19, 2013

Related U.S. Application Data
(60) Provisional application No. 61/373,421, filed on Aug. 13, 2010.

(51) Int. Cl. C07K 16/00; C07K 14/00

(52) U.S. Cl. CPC C07K 16/00; C07K 14/00
USPC 424/133.1; 530/387.3; 536/23.53; 435/254.2; 435/252.3; 435/252.33; 435/254.21; 435/254.23; 435/419; 435/328

(57) ABSTRACT

Provided are monomeric polypeptides comprising variant Fc regions and methods of using them. In certain embodiments, monomeric polypeptides of the invention are fusion proteins. In certain embodiments, monomeric polypeptides of the invention are antibodies.

### 1. CROSS REFERENCE TO RELATED APPLICATIONS

This application claims priority to U.S. Provisional Application No. 61/373,421 filed Aug. 13, 2010, which is incorporated by reference in its entirety.

### 2. REFERENCE TO A SEQUENCE LISTING

This application incorporates by reference a Sequence Listing submitted with this application as text file MED0585 PCT SL.txt created on Aug. 3, 2011 and having a size of 28,672 bytes.

### 3. FIELD OF THE INVENTION

The present invention relates to monomeric polypeptides comprising variant Fc regions and methods of using them.

### 4. BACKGROUND OF THE INVENTION

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, and the heavy chains are linked to each other although the number of disulfide linkages varies between the heavy chains of different immunoglobulin isotypes. Each light chain is comprised of a light chain variable region (abbreviated herein as VL) and a light chain constant region (abbreviated herein as CL). Each heavy chain is comprised of a heavy chain variable region (VH) and a heavy chain constant region (CH) consisting of three domains, CH1, CH2 and CH3. CH1 and CH2, of the heavy chain, are separated from each other by the so-called hinge region. The hinge region normally comprises one or more cysteine residues, which may form disulphide bridges with the cysteine residues of the hinge region of the other heavy chain in the antibody molecule. Antibodies have a variable domain comprising the antigen-specific binding sites and a constant domain which is involved in effector functions.

### 5. SUMMARY OF THE INVENTION

The invention relates to monomeric polypeptides comprising variant Fc regions having one or more amino acid substitutions that inhibit dimer formation of the Fc region. The monomeric polypeptides may additionally comprise a second polypeptide fused to the variant Fc region, such as, for example, a therapeutic protein or an antigen-binding region of an antibody. In exemplary embodiments, the monomeric polypeptide is a monomeric antibody comprising a heavy chain having a variant Fc region and a light chain.

The invention additionally provides formulations comprising a monomeric polypeptide of the invention and a carrier. In one embodiment, the formulation is a therapeutic formulation comprising a pharmaceutically acceptable carrier. Formulations of the invention may be useful for treating a disease/condition and/or preventing and/or alleviating one or more symptoms of a disease/condition in a mammal. Formulations can be administered to a patient in need of such treatment, wherein the formulation can comprise one or more monomeric polypeptides of the invention. In a further embodiment, the formulations can comprise a monomeric polypeptide in combination with other therapeutic agents.

The invention also provides a nucleic acid molecule encoding a monomeric polypeptide of the invention. The invention further provides expression vectors containing a nucleic acid molecule of the invention and host cells transformed with a nucleic acid molecule of the invention. The invention further provides a method of producing a monomeric polypeptide of the invention, comprising culturing a host cell of the invention under conditions suitable for expression of said monomeric polypeptide.

### 6. BRIEF DESCRIPTION OF THE FIGURES

FIG. 1 shows the SEC-MALLS Profile obtained for the wild type IgG4 Fc domain (panel A), the IgG4 single arginine mutants at positions 366 (panel B) and 407 (panel C), and the 366/407 double arginine mutant (panel D). The wild type construct has a molecular weight that is consistent with dimer, while the three mutants have a significantly reduced molecular weight. Time is in minutes on the x-axis and molar mass is in grams per mole on the y-axis

FIG. 2 shows size exclusion chromatograms of a selection of the mutant IgG4 Fc domains analyzed and comparison of the profiles with that obtained for the known wild type dimer (WT). Panel A shows a large number of the traces obtained for those samples deemed to be similar to the wild type dimer (indicated by an arrow), whereas panel B shows a collection of the mutants that show characteristics more common with a monomeric species. Panel C displays the broad range of retention times obtained for the samples, ranging from mutants with an apparent molecular weight larger than 52 kDa to those with a molecular weight consistent with monomer (˜28 kDa).

FIG. 3 shows analytical SEC chromatograms for wild type and T366/Y407 single and double arginine mutant Fc domains for three IgG subclasses. Each trace is labeled and the number in parentheses reflects the retention time in minutes for the centre of the main peak. Panels A and B show IgG1 and 2 Fc domains respectively, with Y407R appearing to be predominantly monomeric for both subclasses with the other mutants showing signs of a mixed population of monomer and dimer. Panel C shows the IgG4 mutants compared to the wild type, with all samples showing a significant shift to the right with a monodisperse distribution indicative of a monomeric sample.

FIG. 4 shows sedimentation velocity analytical ultracentrifugation (SV-AUC) chromatograms for wild type (Panel A), Y349D (Panel B) and T394D (Panel C) hingeless IgG4 Fc domains. The major peak of the wild type construct has an apparent molecular weight that is consistent with the expected mass of the homodimer, the apparent molecular weight of the major peak of the Y349D mutant is lower consistent with monomer-dimer equilibrium and that of the T394D mutant is consistent with a monomer.

FIG. 5 shows the serum concentrations of a wild type IgG4, aglycosylated monovalent IgG4 and glycosylated IgG4 over a period of 16 days. The dotted horizontal line represents the lower limit of quantification.

FIG. 6 shows an alignment of the CH2 (panel A) and CH3 (panel B) regions of the Fc of human IgG1, IgG2, IgG3, IgG4 and mouse IgG1, IgG2a and IgG2b. The numbering of the ruler is according the EU index as set forth in Kabat (Kabat et al. Sequences of Proteins of Immunological Interest, 5th Ed. Public Health Service, National Institutes of Health, Bethesda, Md. (1991)). In addition to the differences between the isotypes shown, there are also allotype differences known in the art which are not represented.

### 7. DETAILED DESCRIPTION

### 7.1 Introduction

The present invention provides monomeric polypeptides comprising variant Fc regions and methods of using them. In certain embodiments, the monomeric polypeptides comprising variant Fc regions of this disclosure may be monomeric antibodies, monomeric antibody fragments or monomeric fusion proteins. The monomeric polypeptides comprising variant Fc regions of this disclosure are also herein referred to as polypeptides of the invention.

Antibodies are stable dimeric proteins. Immunoglobulin heavy chains are joined at the hinge by interchain disulphide bonds and at the CH3 domains by non-covalent interactions. This is sufficient for most IgG subtypes under most conditions to form stable dimeric antibodies. However, IgG4 antibodies are able to form intra as well as interchain disulphide bonds, leading to arm-exchange (i.e., the heavy chains are able to separate and heavy chains from two different antibodies are able to pair to form heterodimeric molecules).

Antibodies have become a major focus area for therapeutic applications, and many antibody drug products have been approved or are in the process of being approved for use as therapeutic drugs. The desired characteristics of therapeutic antibodies may vary according to the specific condition, which is to be treated. For some applications divalent, full length antibodies or divalent antibody fragments are most advantageous whereas for other applications monomeric antibody fragments would be advantageous. Antibodies have a variable domain comprising the antigen-specific binding sites and a constant domain which is involved in effector functions. For some indications, only antigen binding is required, for instance where the therapeutic effect of the antibody is to block interaction between the antigen and one or more specific molecules otherwise capable of binding to the antigen. For other indications, further effects may also be required, such as the ability to induce complement activation, bind Fc receptors, protect from catabolism, recruit immune cells, etc. For such uses, other parts of the antibody molecule, such as the constant Fc region, may be advantageous.

For some indications dimeric antibodies may exhibit undesirable agonistic effects upon binding to the target antigen, even though the antibody works as an antagonist when used as a Fab fragment. In some instances, this effect may be attributed to “cross-linking” of the bivalent antibodies, which in turn promotes target dimerization, which may lead to activation, especially when the target is a receptor. In the case of soluble antigens, dimerization may form undesirable immune complexes. In some indications full length antibodies may be too large to penetrate the target body compartment required and therefore smaller antibody fragments such as monomeric antibodies may be required. In some cases, monovalent binding to an antigen, such as in the case of FcaRI may induce apoptotic signals.

Candidate protein therapeutics may not have optimal pharmacokinetic properties and/or may benefit from effector functions. To address these deficiencies the Fc region of antibody fragments may be fused to protein therapeutics. Addition of an Fc region may enhance effector function of the polypeptide and may alter the pharmacokinetic properties (e.g., half-life) of the polypeptide. In addition, fusion to an Fc region will also result in the formation of dimers of the protein therapeutic. Avoiding dimerization of the Fc regions has the same advantages for protein fusions as discussed for antibodies.

It would be advantageous to develop variant Fc domains that are substantially or fully monomeric that would facilitate the development of monomeric polypeptides for use as therapeutics. Such variant monomeric Fc domains could be fused to therapeutic proteins for the production of monomeric Fc fusion proteins. Alternatively, such variant monomeric Fc domains would permit the development of monovalent antibodies that would avoid the undesirable side effects associated with dimeric antibodies as described above. The present disclosure is based on the identification and characterization of monomeric antibodies having these unique and advantageous features. These monomeric polypeptides are described in detail herein.

### 7.2 Terminology

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the term “Fc region” refers to the constant region of an antibody excluding the first constant region immunoglobulin domain. Thus, Fc region refers to the last two constant region immunoglobulin domains of IgA, IgD, and IgG, and the last three constant region immunoglobulin domains of IgE and IgM, and the flexible hinge N-terminal to these domains. For IgA and IgM, the Fc region may include the J chain. For IgG, the Fc region comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region comprising a hinge region is usually defined to comprise residues E216 to its carboxyl-terminus, wherein the numbering is according to the EU index as set forth in Kabat. As used herein the term “hinge region” refers to that portion of the Fc region stretching from E216-P230 of IgG1, wherein the numbering is according the EU index as set forth in Kabat. The hinge regions of other IgG isotypes may be aligned with the IgG1 sequence by placing the first and last cysteine residues forming inter-heavy chain disulphide bonds in the same positions as show in Table 1 below.

TABLE 1
Alignment of hinge regions of human IgGs

| IgG | 216 | 217 | 218 | 219 | 220 | 221 | | | 222 | 223 | 224 | 225 | 226 | 227 | 228 | | 229 | 230 |
|---|---|---|---|---|---|---|---|---|---|---|---|---|---|---|---|---|---|---|
| hIgG1 | E | P | K | S | C | D | | | K | T | H | T | C | P | P | | C | P |
| hIgG2 | E | R | K | C | C | | | | V | | E | | C | P | P | | C | P |
| hIgG3 | E | L | K | T | P | L | G | D | T | T | H | T | C | P | R | [CPEPKSCDT | C | P |
| | | | | | | | | | | | | | | | | PPPCPR]_{X3} | | |
| hIgG4 | E | S | K | Y | G | | | | | | P | P | C | P | S | | C | P |

As used herein, the terms “antibody” and “antibodies”, also known as immunoglobulins, encompass monoclonal antibodies (including full-length monoclonal antibodies), polyclonal antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g., the antigen binding portion), disulfide-linked Fvs (dsFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km (1, 2 or 3)). Antibodies may be derived from any mammal, including, but not limited to, humans, monkeys, pigs, horses, rabbits, dogs, cats, mice, etc., or other animals such as birds (e.g., chickens).

As used herein, the term “monomeric protein” or “monomeric polypeptide” refers to a protein or polypeptide that comprises a variant Fc region that is fully or substantially monomeric, e.g., at least 50%, 60%, 70%, 75%, 80%, 85%, 90%, 95%, 97%, 98%, 99% or 100% monomeric.

As used herein, the term “monomeric antibody” or “monomeric antibody fragment” refers to an antibody that comprises a variant Fc region that is fully or substantially monomeric, e.g., at least 50%, 60%, 70%, 75%, 80%, 85%, 90%, 95%, 97%, 98%, 99% or 100% monomeric.

### 7.3 Monomeric Polypeptides

In certain aspects, the invention provides polypeptides comprising a variant Fc region having one or more amino acid alterations (e.g., substitutions, deletions or insertions) that inhibit dimer formation of the Fc region. In certain embodiments, the polypeptides of the invention comprising a variant Fc region are substantially monomeric, e.g., at least 70% of the polypeptide of the invention is monomeric in solution. In exemplary embodiments, the polypeptides of the invention comprising a variant Fc region are substantially monomeric, e.g., at least 70% of the polypeptide of the invention is monomeric in a solution having a concentration of between 0.5 mg/ml to 10.0 mg/ml. In other exemplary embodiments, the polypeptides of the invention comprising a variant Fc region are substantially monomeric, e.g., at least 70% of the polypeptide of the invention is monomeric in a solution having a concentration of between 0.5 mg/ml to 1.0 mg/ml. In certain embodiments, at least 50, 60, 70, 75 80, 85, 90, 95, 96, 97, 98, 99 or 100% of the polypeptide of the invention is monomeric in solution. In certain embodiments, at least 50, 60, 70, 75 80, 85, 90, 95, 96, 97, 98, 99 or 100% of the polypeptide of the invention is monomeric in solution having a concentration of between 0.5 mg/ml to 10.0 mg/ml. In certain embodiments, at least 70% of the polypeptide of the invention is monomeric under in vivo conditions. In certain embodiments, at least 50, 60, 70, 75, 80, 85, 90, 95, 96, 97, 98, 99 or 100% of the polypeptide of the invention is monomeric in solution under in vivo conditions. The percent of monomeric polypeptide may be determined by any suitable means known in the art, including, for example, by Size Exchange Chromatography coupled to Multi Angle Laser Light Scattering (SEC-MALLS) and analytical ultracentrifugation (AUC).

The variant Fc region may be derived from any suitable dimeric parent Fc region, including for example, naturally occurring Fc regions, polymorphic Fc region sequences, engineered Fc regions (e.g., having one or more introduced sequence alterations), or chimeric Fc regions, Fc regions from any species, and Fc regions of any antibody isotype. In various embodiments, the variant Fc region may be derived from a parent Fc region from a human, mouse, rat, rabbit, goat, monkey, feline, or canine. In exemplary embodiments, the variant Fc region is derived from a parent Fc region from a human. In various embodiments, the variant Fc region may be derived from a parent Fc region from an IgG, IgE, IgM, IgD, IgA or IgY antibody. Exemplary variant Fc region sequences are derived from the sequence of a parent Fc region of an IgG immunoglobulin, such as, for example, the Fc region of an IgG1, IgG2, IgG3 or IgG4 immunoglobulin. In a specific embodiment, the variant Fc region is a variant of a human IgG1. In another specific embodiment, the variant Fc region is a variant of a human IgG2. In another specific embodiment, the variant Fc region is a variant of a human IgG3. In still another specific embodiment, the variant Fc region is a variant of a human IgG4. In embodiment, the variant Fc region is a variant of a mouse IgG. In a specific embodiment the variant Fc region is a variant of a mouse IgG1. In another specific embodiment, the variant Fc region is a variant of a mouse IgG2a or IgG2b.

In certain embodiments, the variant Fc region comprises one or more amino acid alterations (e.g., substitutions, deletions or insertions) at residues that form the interface between an Fc homodimer. In exemplary embodiments, the variant Fc region comprises one or more alterations of an amino acid that interacts with itself (a self-interacting residue) in the other chain of an Fc homodimer. See for example self-interacting residues indicated in Table 6. In various embodiments, the variant Fc region comprises one or more amino acid alterations in the CH3 interface, near the CH3 interface. In various embodiments, the variant Fc region further comprises one or more amino acid alterations in the hinge region.

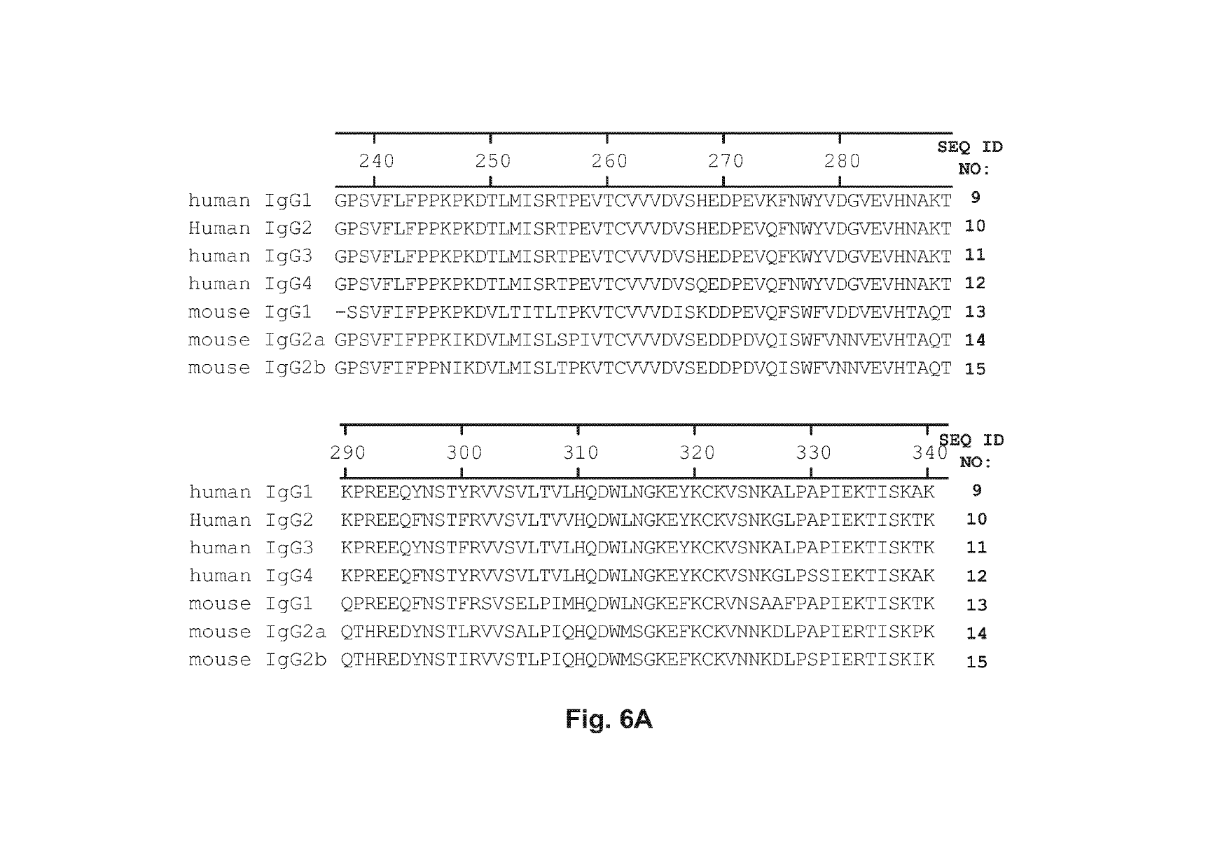

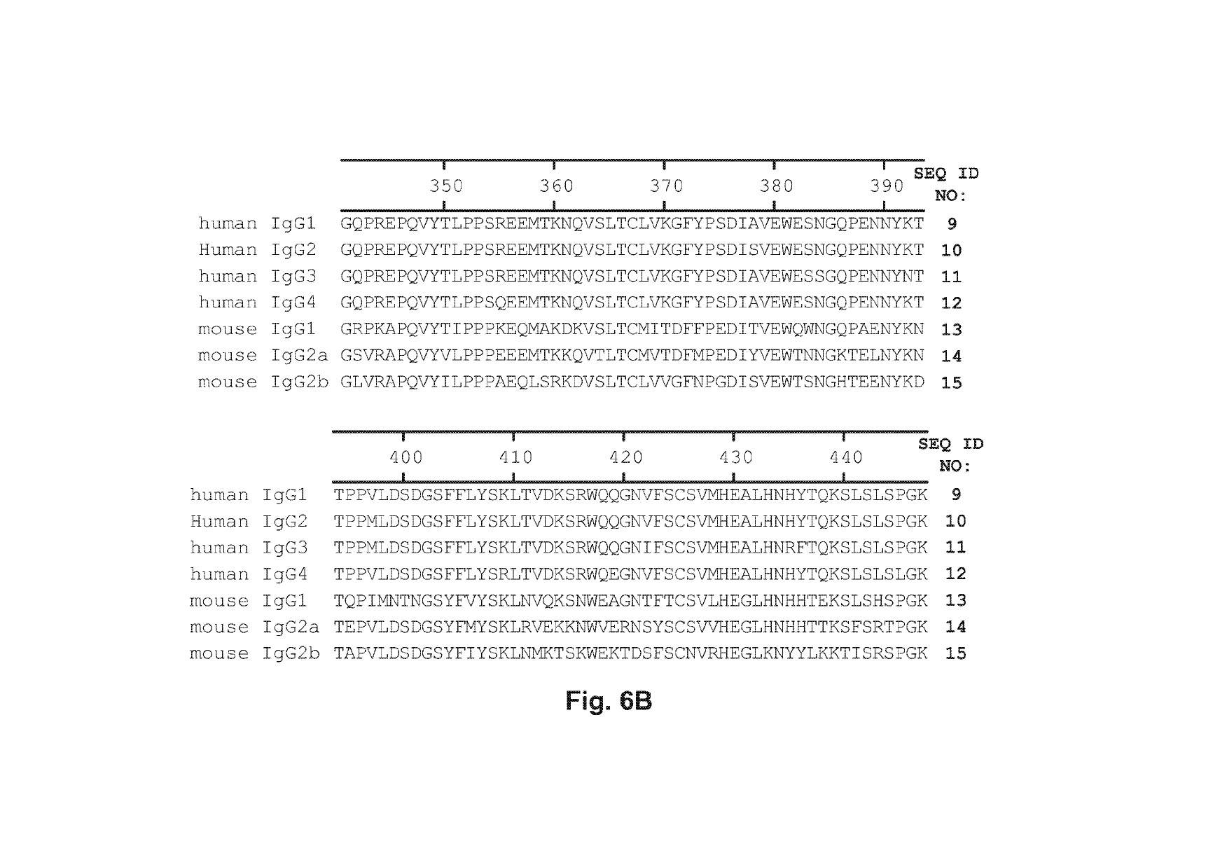

In certain embodiments, the variant Fc region comprises a CH3 interface that is derived from all or a portion of the amino acid sequence of the CH3 interface from a human IgG1, IgG2, IgG3 or IgG4 antibody or the amino acid sequence of the CH3 interface from a mouse IgG2a or IgG2b antibody. The sequences of the CH3 interfaces for such mouse and human antibodies is shown below in Table 2. In certain embodiments, the CH3 interface of the variant Fc region is derived from a sequence that comprises at least 16, 17, 18, 19, 20 or all 21 amino acids of any one of the IgGs as set out in Table 2 below. Allotypic variations are shown at position 356 of hIgG1 and positions 397 and 409 of hIgG3. Amino acids for each immunoglobulin class are aligned and labeled according to Kabat EU numbering as shown in FIG. 6, which refers to the EU index numbering of the human IgG1 Kabat antibody as set forth in Kabat et al., In: Sequences of Proteins of Immunological Interest, US Department of Health and Human Services, 1991.

TABLE 2
Mouse and Human CH3 Interface Sequences

| IgG | 347 | 349 | 350 | 351 | 354 | 356 | 357 | 364 | 366 | 368 | 370 | 392 | 394 | 395 | 397 | 398 | 399 | 405 | 407 | 409 | 439 |
|---|---|---|---|---|---|---|---|---|---|---|---|---|---|---|---|---|---|---|---|---|---|
| hIgG1 | Q | Y | T | L | S | D/E | E | S | T | L | K | K | T | P | V | L | D | F | Y | K | K |
| hIgG2 | Q | Y | T | L | S | E | E | S | T | L | K | K | T | P | M | L | D | F | Y | K | K |
| hIgG3 | Q | Y | T | L | S | E | E | S | T | L | K | N | T | P | M/V | L | D | F | Y | K/R | K |
| hIgG4 | Q | Y | T | L | S | E | E | S | T | L | K | K | T | P | V | L | D | F | Y | R | K |
| mIgG1 | Q | Y | T | I | P | E | Q | S | T | M | T | K | T | Q | I | M | D | F | Y | K | K |
| mIgG2a | Q | Y | V | L | P | E | E | T | T | M | T | K | TE | V | L | D | F | Y | K | K | |
| mIgG2b | Q | Y | I | L | P | E | Q | S | T | L | V | K | T | A | V | L | D | F | Y | K | K |

In Table 2: h = human, m = mouse, hIgG1 Fc from Acc. No. P01857.1; hIgG2 Fc from Acc. No. P01859.2; hIgG3 Fc from Acc. No. BAA11364.1; hIgG4 Fc from Acc. No. P01861.1; mIgG1 Fc from Acc. No. P01868.1, mIgG2a Fc from Acc. No. P01863.1; and m IgG2b Fc from Acc. No. P01867.3.

In certain embodiments, the variant Fc region comprises one or more amino acid substitutions within or close to the CH3 interface of the Fc region. The amino acid substitutions within or close to the CH3 interface may be, for example, substitutions at one or more of the following amino acids according to the Kabat EU numbering system: 347, 349, 350, 351, 352, 354, 356, 357, 360, 362, 364, 366, 368, 370, 390, 392, 393, 394, 395, 396, 397, 398, 399, 400, 405, 406, 407, 408, 409, 411 and 439. In exemplary embodiments, the variant Fc region comprises amino acid substitutions at one or more of the following amino acid positions according to the Kabat EU numbering system: 349, 351, 354, 356, 357, 364, 366, 368, 370, 392, 394, 399, 405, 407, 409, and 439.

In certain embodiments, the variant Fc region comprises one or more amino acid substitutions relative to the parent Fc region sequence that reduce or eliminate homodimerization between two Fc polypeptides, e.g., repelling substitutions. In exemplary embodiments, such repelling substitutions may be made at self-interacting amino acid residues. Examples of suitable repelling substitutions include, for example, substitutions to amino acids having a charged side chain, a large or bulky side chain, or a hydrophilic side chain. For example, an amino acid residue that does not have a positively charged side chain in the parent Fc sequence may be replaced with an amino acid having a positively charged side chain to form the variant Fc region. Exemplary amino acids with positively charged side chains may be selected from: Arginine, Histidine and Lysine. In exemplary embodiments, one or more of the following amino acid positions in a parent Fc region have been substituted with an amino acid having a positively charged side chain to form the variant Fc region: 351, 356, 357, 364, 366, 368, 394, 399, 405 and 407. Alternatively, an amino acid residue that does not have a negatively charged side chain in the parent Fc sequence may be replaced with an amino acid having a negatively charged side chain to form the variant Fc region. Exemplary amino acids having a negatively charged side chain may be selected from: Aspartic acid and Glutamic acid. In exemplary embodiments, one or more of the following amino acid positions in a parent Fc region have been substituted with an amino acid having a negatively charged side chain to form the variant Fc region: 349, 351, 394, 407, and 439. Alternatively, an amino acid residue that does not have a hydrophilic side chain in the parent Fc sequence may be replaced with an amino acid having a hydrophilic side chain to form the variant Fc region. Exemplary amino acids having a hydrophilic side chain may be selected from: Glutamine, Asparagine, Serine and Threonine. In exemplary embodiments, the amino acid at position 366, 405, and 407 in the parent Fc region has been substituted with an amino acid having a hydrophilic side chain to form the variant Fc region. Alternatively, an amino acid residue that does not have a large or bulky side chain in the parent Fc sequence may be replaced with an amino acid having a large or bulky side chain to form the variant Fc region. Exemplary amino acids having a large side chain may be selected from: Tryptophan, Phenylalanine and Tyrosine. In exemplary embodiments, one or more of the following amino acid positions in the parent Fc region have been substituted with an amino acid having a large side chain to form the variant Fc region: 357, 364, 366, 368, and 409.

In certain embodiments, the variant Fc region comprises one or more of the following amino acid substitutions relative to the parent Fc region: (i) amino acid position 405 has been substituted with an amino acid having a positively charged side chain or a hydrophilic side chain, (ii) amino acid position 351 is substituted with an amino acid having a positively charged side chain or a negatively charged side chain, (iii) amino acid position 357 is substituted with an amino acid having a positively charged side chain or a large side chain, (iv) amino acid position 364 is substituted with an amino acid having a positively charged side chain, (v) amino acid position 366 is substituted with an amino acid having a positively charged side chain, (vi) amino acid position 368 is substituted with an amino acid having a positively charged side chain, (vii) amino acid position 394 is substituted with an amino acid having a positively charged side chain or a negatively charged side chain, (viii) amino acid position 399 is substituted with an amino acid having a positively charged side chain, (ix) amino acid position 407 is substituted with an amino acid having a positively charged side chain or a negatively charged side chain, or (x) amino acid position 409 is substituted with an amino acid having a large side chain.

In certain embodiments, the variant Fc region comprises one or more of the following amino acid substitutions relative to the parent Fc region: L351R, L351D, E357R, E357W, S364R, T366R, L368R, T394R, T394D, D399R, F405R, F405Q, Y407R, Y407D, K409W and R409W. In certain embodiments, the variant Fc region comprises one or more amino acid substitutions selected from the group consisting of: Y349D, L351D, L351R, S354D, E356R, D356R, S364R, S364W, T366Q, T366R, T366W, L368R, L368W, T394D, T394R, D399R, F405A, F405Q, Y407A, Y407Q, Y407R, K409R, and K439D.

In certain embodiments, the variant Fc region comprises at least two amino acid substitutions that inhibit dimer formation. In certain embodiments, the variant Fc region comprises at least three amino acid substitutions that inhibit dimer formation. In certain embodiments, the variant Fc region comprises at least 4, 5, 6, 7, 8, 9, 10, 11, 12, 13, 14, 15, 16, 17, 18, 19, 20 or 21 amino acid substitutions that inhibit dimer formation. In certain embodiments, the variant Fc region comprises from 1-21, 1-15, 1-10, 1-5, 1-3, 1-2, 2-21, 2-15, 2-10, 2-5, 2-3, 3-21, 3-15, 3-10, 3-5, 3-4, 5-21, 5-15, 5-10, 5-8, 5-6, 10-21, 10-15, 10-12, 12-15, or 15-20 amino acid substitutions relative to the parent Fc region sequence and the resulting variant Fc region has reduced or eliminated dimer formation relative to the parent Fc region sequence. In certain embodiments, the variant Fc region comprises one or more of the following sets of amino acid substitutions: Y349D/S354D, L351D/T394D, L351D/K409R, L351R/T394R, E356R/D399R, D356R/D399R, S364R/L368R, S364W/L368W, S364W/K409R, T366R/Y407R, T366W/L368W, L368R/K409R, T394D/K409R, D399R/K409R, D399R/K439D, F405A/Y407A, F405Q/Y407Q, L351R/S364R/T394R, and T366Q/F405Q/Y407Q. In certain embodiments, the Fc region comprises any combination of amino acid substitutions.

In certain embodiments, the variant Fc region does not contain a hinge region or comprises a hinge region having one or more mutations including amino acid substitutions, deletions, and/or insertions. For example, at least 1, 2, 3, 4, 5, 6, 7, 8, 9, 10, 11, 12, 14, 15, or more amino acids of the hinge region may be substituted or deleted, or from 1-15, 1-12, 1-10, 1-5, 1-3, 2-15, 2-12, 2-10, 2-5, 5-12, 5-10, or 5-8 amino acids of the hinge region may be substituted or deleted. In certain embodiments, at least one cysteine residue in the hinge region is deleted or substituted with a different amino acid, such as, for example, alanine, serine or glutamine. In an exemplary embodiment, all of the amino acids of the hinge region have been deleted. In other embodiments, the variant Fc region comprises an unaltered hinge region.

In certain embodiments, the variant Fc regions described herein may contain additional modifications that confer an additional desirable function or property to the variant Fc regions having reduced or eliminated dimerization. For example, the variant Fc regions described herein may be combined with other known Fc variants such as those disclosed in Ghetie et al., 1997, Nat. Biotech. 15:637-40; Duncan et al, 1988, Nature 332:563-564; Lund et al., 1991, J. Immunol. 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,083,784; 7,122,637; 7,183,387; 7,217,797; 7,276,585; 7,332,581; 7,355,008; 7,335,742; 7,371,826; 6,821,505; 6,180,377; 7,317,091; 7,355,008; U.S. Publication Nos.: 2002/0147311; 2004/0002587; 2005/0215768; US 2006/0173170; US 2006/024298; 2006/235208; 2007/0135620; 2007/0224188; 2008/0089892; and PCT Publication Nos.: WO 94/29351; and WO 99/58572.

Because Fc receptors (FcR) typically bind both copies of the Fc region in the full-length antibody, the variant Fc regions described herein are generally unlikely to retain the function of antibody-dependent cytotoxicity (ADCC). This lack of FcR binding may be useful in antibody or Fc fusion proteins in cases where Fc receptor stimulation is not desired. However, variant Fc regions from IgA antibodies may still bind to their FcaR since the receptor binds to the Ca2/Ca3 interface within a single Fc chain (e.g., an Fc monomer). In addition, the neo-natal Fc receptor (FcRn) only binds one Fc monomer suggesting that the variant Fc regions of the present invention may largely retain FcRn binding.

In certain embodiments, the variant Fc regions described herein do not bind one or more FcRs and do not have antibody-dependent cellular cytotoxicity (ADCC), complement dependent cytotoxicity (CDC), and/or antibody dependent cell-mediated phagocytosis (ADCP) activity. In other embodiments, the variant Fc regions described herein have additional modifications that result in a decrease or increase of FcaR binding, FcRn binding, antibody-dependent cellular cytotoxicity (ADCC), or antibody dependent cell-mediated phagocytosis (ADCP).

In certain embodiments, the variant Fc regions described herein comprise additional modifications that increase the binding affinity of the variant Fc region for FcRn, which results in an increase in the serum half-life of a polypeptide containing the variant Fc region. For example, monomeric polypeptides of the invention with increased half-lives may be generated by modifying amino acid residues identified as involved in the interaction between the Fc and the FcRn receptor (see, for examples, U.S. Pat. Nos. 6,821,505 and 7,083,784; and WO 09/058,492). In certain embodiments, the variant Fc regions described herein further comprise one or more amino acid substitutions selected from the group consisting of: M252Y, S254T, T256E, P257N, P257L, M428L, N434S, and N434Y. In other embodiment, the variant Fc regions described herein further comprise one or more of the following sets of amino acid substitutions M252Y/S254T/T256E, P257L/M434Y, P257N/M434Y, and M428L/N434S. In a specific embodiment, the variant Fc regions described herein further comprise the amino acid substitutions M252Y/S254T/T256E. The term “polypeptide half-life” as used herein means a pharmacokinetic property of a polypeptide that is a measure of the mean survival time of polypeptide molecules following their administration. Polypeptide half-life can be expressed as the time required to eliminate 50 percent of a known quantity of protein from the patient's body (or other mammal) or a specific compartment thereof, for example, as measured in serum, i.e., circulating half-life, or in other tissues. Half-life may vary from one polypeptide or class of polypeptides to another. In general, an increase in polypeptide half-life results in an increase in mean residence time (MRT) in circulation for the polypeptide administered. The increase in half-life allows for the reduction in amount of drug given to a patient as well as reducing the frequency of administration.

In certain embodiments, a variant Fc region described herein exhibits increased or decreased affinity for a FcaR and/or FcRn that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or is between 2 fold and 10 fold, or between 5 fold and 50 fold, or between 25 fold and 100 fold, or between 75 fold and 200 fold, or between 100 and 200 fold, more or less than the parent Fc region. In another embodiment, a variant Fc region described herein exhibits affinities for FcaR and/or FcRn that are at least 90%, at least 80%, at least 70%, at least 60%, at least 50%, at least 40%, at least 30%, at least 20%, at least 10%, or at least 5% more or less than the parent Fc region. In certain embodiments, a variant Fc region of the invention has increased affinity for FcaR and/or FcRn. In other embodiments, a variant Fc region of the invention has decreased affinity for FcaR and/or FcRn.

In certain embodiments, the sequence of a variant Fc region of the invention shares substantial amino acid sequence identity with the parent Fc region. For example, the amino acid sequence of a variant Fc region of the invention may have at least 50%, 60%, 70%, 75%, 80%, 85%, 90%, 95%, 96%, 97%, 98%, or 99% identity with the amino acid sequence of the parent Fc region.

In certain embodiments, the monomeric polypeptides of the invention can be purified by isolation/purification methods for proteins generally known in the field of protein chemistry and as further described herein. The purified monomeric polypeptide is preferably at least 85% pure, more preferably at least 95% pure, and most preferably at least 98% pure. Regardless of the exact numerical value of the purity, the polypeptide is sufficiently pure for use as a pharmaceutical product.

In certain embodiments, polypeptides comprising a variant Fc region as described herein may be glycosylated or aglycosyl. In certain embodiments, the portion of the polypeptide comprising the variant Fc region is glycosylated or aglycosyl. The variant Fc region may comprise a native glycosylation pattern or an altered glycosylation pattern. An altered glycosylation pattern can be accomplished by, for example, altering one or more sites of glycosylation within the Fc region sequence. For example, one or more amino acid substitutions can be made that result in elimination of one or more glycosylation sites to thereby eliminate glycosylation at that site (e.g., Asparagine 297 of IgG). Such aglycosylated polypeptides comprising a variant Fc region may be produced in bacterial cells which lack the necessary glycosylation machinery.

Addition of sialic acid to the oligosaccharides on an Fc region can enhance the anti-inflammatory activity and alter the cytotoxicity of such molecules (Keneko et al., Science, 2006, 313:670-673; Scallon et al., Mol. Immuno. 2007 March; 44(7):1524-34). Therefore, a polypeptide comprising a variant Fc region can be modified with an appropriate sialylation profile for a particular therapeutic application (US Publication No. 2009/0004179 and International Publication No. WO 2007/005786). In one embodiment, the variant Fc regions described herein comprise an altered sialylation profile compared to the native Fc region. In one embodiment, the variant Fc regions described herein comprise an increased sialylation profile compared to the native Fc region. In another embodiment, the variant Fc regions described herein comprise a decreased sialylation profile compared to the native Fc region.

### 7.3.1 Fc Fusion Proteins

In certain embodiments, the monomeric polypeptides of the invention are Fc fusion proteins, e.g., polypeptides comprising a variant Fc region as described herein conjugated to one or more heterologous protein portions. Any desired heterologous polypeptide may be fused to the variant Fc region to form the Fc fusion protein, including, for example, therapeutic proteins, antibody fragments lacking an Fc region and protein scaffolds. In exemplary embodiments, the Fc region is fused to a heterologous polypeptide for which it is desirable to increase the size, solubility, expression yield, and/or serum half-life of the polypeptide. In certain embodiments, the Fc region is fused to a heterologous polypeptide as a tag for purification and/or detection of the heterologous polypeptide. In exemplary embodiments, the Fc fusion proteins of the invention are substantially monomeric, e.g., at least 50%, 60%, 70%, 75%, 80%, 85%, 90%, 95%, 97%, 98%, 99% or 100% of the Fc fusion protein is monomeric in solution.

In certain embodiments, a variant Fc region described herein may be fused or otherwise linked at the N and/or C-terminus to one or more heterologous polypeptide(s). The variant Fc region may be linked to a heterologous polypeptide directly or via a chemical or amino acid linker by any suitable means known in the art including, for example, chemical conjugation, chemical cross-linking, or genetic fusion. Preferably, a variant Fc region is linked to a heterologous polypeptide sequence such that the Fc domain and heterologous polypeptide portion are properly folded, and the heterologous polypeptide portion(s) retain biological activity.

Fc fusions of the invention may be used when monovalency is desired for obtaining a therapeutic effect. For example, Fc fusions of the invention may be used if there are concerns that bivalency of an Fc fusion might induce receptor dimerization resulting in an undesired modulation in a signaling pathway. Fc fusions of the invention may also be desirable when it is preferred that a therapeutic Fc Fusion effects its therapeutic action without inducing immune system-mediated activities, such as the effector functions, ADCC, phagocytosis and CDC.

The Fc fusions of the present invention have numerous in vitro and in vivo diagnostic and therapeutic utilities involving the diagnosis and treatment of disorders. The invention does not relate to Fc fusion proteins incorporating any specific heterologous protein portion, as according to the invention the monovalent polypeptide described in the present specification may incorporate any heterologous protein portion. The specific utility of an Fc fusion protein of the invention will be dependent on the specific heterologous protein portion. The selection of heterologous proteins may be based on the therapeutic value and/or the advantages of administering a monovalent form of the heterologous protein. Such considerations are within the skills of a person of skill in the art. An Fc fusion protein of the invention may be used as an antagonist and/or inhibitor to partially or fully block the activity of a molecule. In a specific embodiment, an Fc fusion protein of the invention comprises a receptor binding portion of a ligand which may bind to the receptor and block or interfere with the binding of the native ligand to the receptor thereby inhibiting the corresponding signaling pathway. In other embodiments, an Fc fusion protein of the invention comprises a ligand binding domain of a receptor which may bind native ligand thereby preventing the ligand from binding to the native receptor thereby inhibiting the corresponding signaling pathway. In still other embodiments, a monovalent polypeptide of the invention comprises a heterologous molecule having therapeutic efficacy for which an extended half-life is desired.

In certain embodiments, variant Fc regions may be used as tags to facilitate purification of one or more heterologous polypeptides. Fc Fusion proteins of the invention may be purified using any suitable method known in the art for isolating polypeptides comprising an Fc-domain including, for example, chromatograph techniques such as ion exchange, size exclusion, hydrophobic interaction chromatography, as well as use of protein A and/or protein G, and/or anti-Fc antibodies, or combinations thereof. In general, purification of Fc-tagged protein from medium or cell lysates involves using Protein A or Protein G coupled to a resin (e.g., agarose or sepharose beads). The purification can be performed, for example, in batch form, by incubating a Protein A or Protein G resin in solution with the Fc-tagged protein followed by a centrifugation step to isolate resin from the soluble fraction, or by passing a solution of the Fc-tagged protein through a column containing a Protein A or Protein G resin. Elution of Fc-tagged proteins from Protein A or Protein G may be preformed by any suitable method including, for example, incubating the Fc-bound resin in buffers of varying isotonicity and/or pH. Fc-tagged polypeptides may be further purified using various techniques including, for example, ion exchange, size exclusion, hydrophobic interaction chromatography, or combinations thereof.

In certain embodiments, variant Fc regions may be used as tags to facilitate detection of one or more heterologous polypeptides. Fc Fusion proteins of the disclosure may be detected using any suitable method known in the art for identifying polypeptides comprising an Fc-domain including, for example, use of labeled Fc-binding proteins such as Protein A, Protein G, and/or anti-Fc antibodies. Such Fc-binding proteins may be conjugated to any suitable detection reagent including, for example, a chromophore, a fluorophore, a fluorescent moiety, a phosphorescent dye, a tandem dye, a hapten, biotin, an enzyme-conjugate, and/or a radioisotope (see, e.g., U.S. Pat. Application No. 2009/0124511, the teachings of which are incorporated herein by reference). Following incubation with one or more labeled Fc-binding proteins, proteins tagged with a variant Fc region of the disclosure may be identified using one or more immunodetection techniques well known in the art including, for example, immunofluorescence microscopy, flow cytometry, immunoprecipitation, Western blotting, ELISA, and/or autoradiogram. In certain aspects, such labeled Fc-binding proteins may also be used to facilitate purification of Fc-tagged proteins of the disclosure. For example, Fc-tagged proteins may be conjugated to one or more fluorescently-labeled anti-Fc antibodies and then isolated using various fluorescence-activated cell sorting methods known in the art.

Exemplary categories of heterologous proteins include, but are not limited to, enzymes, growth factors (such as, for example, transforming growth factors, e.g., TGF-alpha, TGF-beta, TGF-beta2, TGF-beta3), therapeutic proteins (e.g., erythropoietin (EPO), interferon (e.g., IFN-γ), or tumor necrosis factor (e.g., TNF-α)), cytokines, extracellular domains of transmembrane receptors, receptor ligands, antibody fragments lacking a complete Fc region (e.g., an antigen binding fragment of an antibody), or a non-immunoglobulin target binding scaffold.

In certain embodiments, the heterologous protein is an antigen binding portion of an antibody. The antigen-binding portion of an antibody comprises one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a domain antibody (dAb) fragment (Ward et al., (1989) Nature 341:544-546), which consists of a VH domain; (vi) an isolated complementarity determining region (CDR); (vii) a single chain Fv (scFv) consisting of the two domains of the Fv fragment, VL and VH, joined by a synthetic linker that enables them to be made as a single protein chain in which the VL and VH regions pair to form monovalent molecules (see e.g., Bird et al. (1988) Science 242:423-426; and Huston et al. (1988) Proc. Natl. Acad. Sci. USA 85:5879-5883)); (viii) vaccibodies (see U.S. Publication No. 2004/0253238); and (ix) bispecific or monospecific linear antibodies consisting of a pair of tandem Fd segments (V_{H}-C_{H1}-V_{H}-C_{H1}) which form a pair of antigen-binding regions (see Zapata et al., Protein Eng., 8(10):1057-1062 (1995) and U.S. Pat. No. 5,641,870).

Antibody fragments may be obtained using conventional techniques known to those of skill in the art, and the fragments may be screened for utility in the same manner as are intact antibodies. Traditionally, antibody fragments were derived via proteolytic digestion of intact antibodies using techniques well known in the art. However, antibody fragments can now be produced directly by recombinant host cells. Fab, Fv and scFv antibody fragments can all be expressed in and secreted from E. coli, thus allowing the facile production of large amounts of these fragments. In one embodiment, the antibody fragments can be isolated from the antibody phage libraries discussed below. Alternatively, Fab′-SH fragments can also be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Better et al., Science 240:1041-1043 (1988). Examples of techniques which can be used to produce single-chain Fvs and antibodies include those described in U.S. Pat. Nos. 4,946,778 and 5,258,498. Examples of domain antibodies include, but are not limited to, those available from Domantis that are specific to therapeutic targets (see, for example, WO04/058821; WO04/081026; WO04/003019; WO03/002609; U.S. Pat. Nos. 6,291,158; 6,582,915; 6,696,245; and 6,593,081). Commercially available libraries of domain antibodies can be used to identify monoclonal domain antibodies.

In certain embodiments, the Fc fusion proteins of the invention comprise a variant Fc region conjugated to a heterologous polypeptide that is a non-immunoglobulin target binding scaffold. Non-immunoglobulin target binding scaffolds are typically derived from a reference protein by having a mutated amino acid sequence. Exemplary non-immunoglobulin target binding scaffolds may be derived from an antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin. Other suitable protein scaffolds are described in Wurch et al. (2008) Current Pharmaceutical Biotechnology, 9:502, incorporated by reference herein.

Fc fusion proteins may be constructed in any suitable configuration. In certain embodiments, the C-terminus of a variant Fc region can be linked to the N-terminus of a heterologous protein. Alternatively, the C-terminus of a heterologous protein can be linked to the N-terminus of a variant Fc region. In certain embodiments, the heterologous protein can be linked to an exposed internal (non-terminus) residue of the variant Fc region or the variant Fc region can be linked to an exposed internal (non-terminus) residue of the heterologous protein. In further embodiments, any combination of the variant Fc-heterologous protein configurations can be employed, thereby resulting in a variant Fc:heterologous protein ratio that is greater than 1:1 (e.g., two variant Fc molecules to one heterologous protein).

The variant Fc region and the heterologous protein may be conjugated directly to each other or they may be conjugated indirectly using a linker sequence. In exemplary embodiments, the linker sequence separates the variant Fc region and the heterologous protein by a distance sufficient to ensure that each portion properly folds into its proper secondary and tertiary structures. Suitable linker sequences may have one or more of the following properties: (1) able to adopt a flexible extended conformation, (2) does not exhibit a propensity for developing an ordered secondary structure which could interact with the functional domains of the variant Fc polypeptide or the heterologous protein, and/or (3) has minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence. In a specific embodiment, a linker sequence length of about 15 amino acids can be used to provide a suitable separation of functional protein domains, although longer or shorter linker sequences may also be used. The length of the linker sequence separating the variant Fc region and the heterologous protein can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids or from about 10 to about 20 amino acids.

In certain embodiments, a variant Fc region may be fused to one or more heterologous polypeptides via a cleavable linker. A variety of cleavable linkers are known to those of skill in the art (see, e.g., U.S. Pat. Nos. 4,618,492; 4,542,225; 4,625,014; 5,141,648; and 4,671,958, the teachings of which are incorporated herein by reference). The mechanisms for release of an agent from these linker groups include, for example, irradiation of a photo-labile bond, acid-catalyzed hydrolysis, and cleavage by proteolytic enzymes. In exemplary embodiments, a variant Fc region of the disclosure used as a tag to facilitate purification and/or detection of a heterologous polypeptide may be removed from the heterologous polypeptide following purification and/or detection by chemical or enzymatic cleavage of a cleavable linker

In certain embodiments, the Fc fusion proteins of the present invention comprising a variant Fc region and a heterologous polypeptide can be generated using well-known cross-linking reagents and protocols. For example, there are a large number of chemical cross-linking agents that are known to those skilled in the art and useful for cross-linking the variant Fc region with a heterologous protein. For example, suitable cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP). Cross-linking agents having N-hydroxysuccinimide moieties can be obtained as the N-hydroxysulfosuccinimide analogs, which generally have greater water solubility. In addition, cross-linking agents having disulfide bridges within the linking chain can be synthesized instead as the alkyl derivatives so as to reduce the amount of linker cleavage in vivo. Other suitable cross-linking agents include homobifunctional and photoreactive cross-linkers. Disuccinimidyl subcrate (DSS), bismaleimidohexane (BMH) and dimethylpimelimidate.2 HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers. For a recent review of protein coupling techniques, see Means et al. (1990) Bioconjugate Chemistry. 1:2-12, incorporated by reference herein.

In certain embodiments, Fc fusion proteins of the invention can be produced using standard protein chemistry techniques such as those described in Bodansky, M. Principles of Peptide Synthesis, Springer Verlag, Berlin (1993) and Grant G. A. (ed.), Synthetic Peptides: A User's Guide, W.H. Freeman and Company, New York (1992). Automated peptide synthesizers suitable for production of the Fc fusion proteins described herein are commercially available (e.g., Advanced ChemTech Model 396; Milligen/Biosearch 9600).

In any of the foregoing methods of cross-linking for chemical conjugation of a variant Fc region to a heterologous polypeptide, a cleavable domain or cleavable linker can be used. Cleavage will allow separation of the heterologous polypeptide and the variant Fc region. For example, following penetration of a cell by an Fc fusion protein, cleavage of the cleavable linker would allow separation of the variant Fc region from the heterologous polypeptide.

In certain embodiments, the Fc fusion proteins of the present invention can be generated as a recombinant fusion protein containing a variant Fc region and a heterologous polypeptide expressed as one contiguous polypeptide chain. Such fusion proteins are referred to herein as recombinantly conjugated. In preparing such fusion proteins, a fusion gene is constructed comprising nucleic acids which encode a variant Fc region and a heterologous polypeptide, and optionally, a peptide linker sequence to connect the variant Fc region and the heterologous polypeptide. The use of recombinant DNA techniques to create a fusion gene, with the translational product being the desired fusion protein, is well known in the art. Examples of methods for producing fusion proteins are described in PCT applications PCT/US87/02968, PCT/US89/03587 and PCT/US90/07335, as well as Traunecker et al. (1989) Nature 339:68, incorporated by reference herein. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992). The Fc fusion protein encoded by the fusion gene may be recombinantly produced using various expression systems as is well known in the art (also see below).

### 7.3.2 Monomeric Antibodies

In certain embodiments, the monomeric polypeptides of the invention are monomeric antibodies, e.g., antibodies or antibody fragments comprising a variant Fc region, wherein the antibodies or antibody fragments are substantially monomeric and immunospecifically bind to a target. In an exemplary embodiment, a monomeric antibody comprises a heavy chain having a variant Fc region as described herein and a light chain, wherein the antibody is substantially monomeric. Monomeric antibodies may be monomeric forms of any type of antibody including, for example, monomeric forms of monoclonal antibodies, chimeric antibodies, nonhuman antibodies, humanized antibodies, or fully human antibodies, or fragments of any of the foregoing that include a variant Fc region. Monomeric antibodies or fragments thereof comprising a variant Fc region may be derived from any source including, for example, humans, monkeys, pigs, horses, rabbits, dogs, cats, mice, chickens, etc., and may be of any isotype.

Monomeric antibodies comprising a variant Fc region as described herein may be made by any suitable means. For example, the sequence of the Fc region of the antibody or antibody fragment may be modified to introduce the Fc region sequence variants as described herein that lead to an increase in the monomeric form of the Fc region. Alternatively, all or a substantial portion of the parent Fc region of the antibody or fragment may be replaced with the sequence of a variant Fc region as described herein. When replacing the parent Fc region of the antibody to introduce a variant Fc region, the replacement Fc region may be from an antibody of the same species and/or isotype or from an antibody of a different species and/or isotype, thereby forming a chimeric antibody. For example, the parent Fc region of a human IgG4 antibody may be replaced with a variant human IgG4 Fc region to form a monomeric human antibody. Alternatively, the parent Fc region of a mouse IgG antibody may be replaced with a variant Fc region from a human IgG antibody thereby forming a monomeric chimeric antibody. Such Fc modifications may be made using standard recombinant DNA techniques as known in the art and as further described herein.

Monomeric antibodies of the invention may be used when monovalency is desired for obtaining a therapeutic effect. For example, a monomeric antibody may be used if there are concerns that bivalency of an antibody might induce a target cell to undergo antigenic modulation. Monomeric antibodies of the invention may also be desirable when it is preferred that a therapeutic antibody effects its therapeutic action without involving immune system-mediated activities, such as the effector functions, ADCC, phagocytosis and CDC. Accordingly, the monomeric antibodies of the present invention have numerous in vitro and in vivo diagnostic and therapeutic utilities involving the diagnosis and treatment of disorders.

It will be understood, that the invention does not relate to monomeric antibodies directed at any specific antigen, as according to the invention the monomeric antibodies described in the present specification may bind to any antigen. The specific utility of a monomeric antibody of the invention will be dependent on the specific target antigen. The selection of a target antigen may be based on the therapeutic value and/or the advantages of administering a monovalent form of the antibody specific for the target antigen. Such considerations are within the skills of a person of skill in the art. A monomeric antibody of the invention may be used as an antagonist and/or inhibitor to partially or fully block the specific antigen activity in vitro, ex vivo and/or in vivo. In a specific embodiment, a monomeric antibody of the invention is specific to a ligand antigen, and inhibits the antigen activity by blocking or interfering with the ligand-receptor interaction involving the ligand antigen, thereby inhibiting the corresponding signaling pathway and other molecular or cellular events. In other embodiments, a monomeric antibody of the invention is specific to a receptor antigen, which may be activated by contact with a ligand, and inhibits the antigen activity by blocking or interfering with the ligand-receptor interaction, thereby inhibiting the corresponding signaling pathway and other molecular or cellular events.

Monomeric antibodies as described herein may immunospecifically interact with any desired target depending on the intended use of the monomeric antibody. For example, monomeric antibodies may bind to a target such as, for example, a cell surface receptor, a cancer antigen, a cytokine, an enzyme, etc. Monomeric antibodies may be derived from existing antibodies, including commercially available forms of antibodies, or from newly isolated antibodies. Exemplary commercially available antibodies include, but are not limited to, Humira®, Remicade®, Simponi®, Rituxan®, Herceptin®, and the like. Methods for making various types of antibodies are well known in the art and are further described below.

In certain embodiments, the monomeric antibody or antibody fragment comprising a variant Fc region immunospecifically binds to a target with a K_{D} of less than 250 nanomolar. In certain embodiments, the K_{D} is less than 100, less than 50, less than 25, or less than 1 nanomolar. In certain embodiments, the K_{D} under these conditions is less than 900, less than 800, less than 700, less than 600, less than 500, less than 400, less than 300, less than 200, or less than 100 picomolar. In certain embodiments, the monomeric antibody or antibody fragment comprising a variant Fc region immunospecifically inhibits a target with a IC₅₀ of less than 250 nanomolar. In certain embodiments, the IC₅₀ is less than 100, less than 50, less than 25, or less than 1 nanomolar. In certain embodiments, the IC₅₀ under these conditions is less than 900, less than 800, less than 700, less than 600, less than 500, less than 400, less than 300, less than 200, or less than 100 picomlar. In certain embodiments, the K_{d} and/or IC₅₀ for a monomeric antibody may be measured using any method known in the art, including, for example, by BIACORE™ affinity data, cell binding, standard ELISA or standard Flow Cytometry assays.

In certain embodiments, the binding affinity of the monomeric antibody is substantially the same as the binding affinity of the parent antibody, e.g., the introduction of one or more sequence variations in the Fc region to produce a variant Fc region as described herein has little to no effect on the binding affinity of the antibody. For example, the introduction of sequence variations in the Fc region of the antibody to produce a monomeric antibody results in less than a 50%, 40%, 30%, 25%, 20%, 15%, 10%, 8%, 6%, 5%, 4%, 3%, 2%, or 1% change in the binding affinity of the antibody for the target. Alternatively, the introduction of sequence variations in the Fc region of the antibody to produce a monomeric antibody results in less than a 10-fold, 8-fold, 5-fold, 4-fold, 3-fold, or 2-fold change in the binding affinity of the antibody for the target. In certain embodiments, the monomeric antibody maintains at least 50%, 60%, 70%, 75%, 80%, 85%, 90%, 95%, 96%, 97%, 98%, or 99% of the binding affinity of the parent antibody for its target. In certain embodiments, the binding affinity of the monomeric antibody for the target is within 10-fold, 8-fold, 5-fold, 4-fold, 3-fold, or 2-fold of the binding affinity of the parent antibody for the same target.

In one embodiment, the monomeric antibodies of the invention are monoclonal antibodies or fragments thereof that contain a variant Fc region as described herein. Monoclonal antibodies can be prepared using a wide variety of techniques known in the art including the use of hybridoma (Kohler et al., Nature, 256:495 (1975); Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), recombinant, and phage display technologies, or a combination thereof. The term “monoclonal antibody” as used herein refers to an antibody obtained from a population of substantially homogeneous or isolated antibodies, e.g., the individual antibodies comprising the population are identical except for possible naturally occurring mutations that may be present in minor amounts. Monoclonal antibodies are highly specific, being directed against a single antigenic site or multiple antigenic sites in the case of multispecific engineered antibodies. Furthermore, in contrast to polyclonal antibody preparations which include different antibodies directed against different determinants (epitopes), each monoclonal antibody is directed against the same determinant on the antigen. In addition to their specificity, monoclonal antibodies are advantageous in that they may be synthesized uncontaminated by other antibodies. The modifier “monoclonal” is not to be construed as requiring production of the antibody by any particular method.

Methods for producing and screening for monoclonal antibodies using hybridoma technology are routine and well known in the art. See e.g., Goding, Monoclonal Antibodies: Principles and Practice, pp. 59-103 (Academic Press, 1986); Kozbor, J. Immunol., 133:3001 (1984); and Brodeur et al., Monoclonal Antibody Production Techniques and Applications, pp. 51-63 (Marcel Dekker, Inc., New York, 1987). Additionally, methods for producing monoclonal antibodies using antibody phage libraries are routine and well known in the art. See e.g., McCafferty et al., Nature, 348:552-554 (1990); and Clackson et al., Nature, 352:624-628 (1991) and Marks et al., J. Mol. Biol., 222:581-597 (1991). In addition to commercially available kits for generating phage display libraries (e.g., the Pharmacia Recombinant Phage Antibody System, catalog no. 27-9400-01; and the Stratagene SURFZAP™ phage display kit, catalog no. 240612), examples of methods and reagents for use in generating and screening antibody display libraries can be found in, for example, U.S. Pat. Nos. 6,248,516; U.S. Pat. Nos. 6,545,142; 6,291,158; 6,291,1591; 6,291,160; 6,291,161; 6,680,192; 5,969,108; 6,172,197; 6,806,079; 5,885,793; 6,521,404; 6,544,731; 6,555,313; 6,593,081; 6,582,915; 7,195,866.

In one embodiment, the monomeric antibodies of the invention are humanized antibodies, chimeric antibodies, or fragments thereof that contain a variant Fc region as described herein. Humanized antibodies are antibody molecules derived from a non-human species antibody (also referred to herein as a donor antibody) that binds the desired antigen. Humanized antibodies have one or more complementarity determining regions (CDRs) from the donor antibody and one or more framework regions from a human immunoglobulin molecule (also referred to herein as an acceptor antibody). Often, framework residues in the human framework regions will be substituted with the corresponding residue from the donor antibody to alter, preferably improve, antigen binding and/or reduce immunogenicity. These framework substitutions are identified by methods well known in the art, e.g., by modeling of the interactions of the CDR and framework residues to identify framework residues important for antigen binding and sequence comparison to identify unusual framework residues at particular positions. (See, e.g., Riechmann et al., Nature 332:323 (1988)). In practice, and in certain embodiments, humanized antibodies are typically human antibodies in which some hypervariable region residues and possibly some FR residues are substituted by residues from analogous sites in the donor antibody. In alternative embodiments, the FR residues are fully human residues.

Humanization can be performed following the method of Winter and co-workers (Jones et al., Nature, 321:522-525 (1986); Reichmann et al., Supra; Verhoeyen et al., Science, 239:1534-1536 (1988)), by substituting hypervariable region sequences for the corresponding sequences of a human antibody. Specifically, humanized antibodies may be prepared by methods well known in the art including CDR grafting approaches (see, e.g., U.S. Pat. No. 6,548,640), veneering or resurfacing (U.S. Pat. Nos. 5,639,641 and 6,797,492; Studnicka et al., Protein Engineering 7(6):805-814 (1994); Roguska. et al., PNAS 91:969-973 (1994)), chain shuffling strategies (see e.g., U.S. Pat. No. 5,565,332; Rader et al., Proc. Natl. Acad. Sci. USA (1998) 95:8910-8915), molecular modeling strategies (U.S. Pat. No. 5,639,641), and the like. These general approaches may be combined with standard mutagenesis and recombinant synthesis techniques to produce monomeric humanized antibodies with desired properties.

By definition, humanized antibodies are chimeric antibodies. Chimeric antibodies are antibodies in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity (e.g., Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

In one embodiment, the monomeric antibodies of the invention are human antibodies or fragments thereof that contain a variant Fc region as described herein. Human antibodies avoid some of the problems associated with antibodies that possess murine or rat variable and/or constant region sequences. The presence of such murine or rat derived sequences can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

Human antibodies can be generated using methods well known in the art. For example, it is now possible to produce transgenic animals (e.g., mice) that are capable, upon immunization, of producing a full repertoire of human antibodies in the absence of endogenous immunoglobulin production. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. The use of XENOMOUSE® strains of mice for production of human antibodies has been described. See Mendez et al. Nature Genetics 15:146-156 (1997) and Green and Jakobovits J. Exp. Med. 188:483-495 (1998). The XENOMOUSE® strains are available from Amgen, Inc. (Fremont, Calif.). The production of the XENOMOUSE® strains of mice and antibodies produced in those mice is further discussed in U.S. Pat. Nos. 6,673,986; 7,049,426; 6,833,268; 6,162,963, 6,150,584, 6,114,598, 6,075,181, 6,657,103; 6,713,610 and 5,939,598; US Publication Nos. 2004/0010810; 2003/0229905; 2004/0093622; 2005/0054055; 2005/0076395; and 2006/0040363. In an alternative approach, others, including GenPharm International, Inc., have utilized a “minilocus” approach. This approach is described in U.S. Pat. Nos. 5,545,807; 5,545,806; 5,625,825; 5,625,126; 5,633,425; 5,661,016; 5,770,429; 5,789,650; 5,814,318; 5,877,397; 5,874,299; 6,255,458; 5,591,669; 6,023,010; 5,612,205; 5,721,367; 5,789,215; 5,643,763; and 5,981,175. Kirin has also demonstrated the generation of human antibodies from mice in which large pieces of chromosomes, or entire chromosomes, have been introduced through microcell fusion. See U.S. Pat. No. 6,632,976. Additionally, KM™ mice, which are the result of cross-breeding of Kirin's Tc mice with Medarex's minilocus (Humab) mice, have been generated. These mice possess the human IgH transchromosome of the Kirin mice and the kappa chain transgene of the Genpharm mice (Ishida et al., Cloning Stem Cells, (2002) 4:91-102). Human antibodies can also be derived by in vitro methods. Suitable examples include but are not limited to phage display (MedImmune (formerly CAT), Morphosys, Dyax, Biosite/Medarex, Xoma, Symphogen, Alexion (formerly Proliferon), Affimed) ribosome display (MedImmune (formerly CAT)), yeast display, and the like. Phage display technology (See e.g., U.S. Pat. No. 5,969,108) can be used to produce human antibodies and antibody fragments in vitro, from immunoglobulin variable (V) domain gene repertoires from unimmunized donors. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. See e.g., Clackson et al., Nature, 352:624-628 (1991); Marks et al., J. Mol. Biol. 222:581-597 (1991); Griffith et al., EMBO J. 12:725-734 (1993); and U.S. Pat. Nos. 5,565,332 and 5,573,905. As discussed above, human antibodies may also be generated by in vitro activated B cells (see U.S. Pat. Nos. 5,567,610 and 5,229,275).

### 7.3.3 Heterologous Proteins and Antigens

Generally, when the monomeric polypeptide of the invention is an antibody or comprises an antigen binding portion, the monomeric polypeptide of the invention specifically binds an antigen of interest. In one embodiment, a monomeric polypeptide of the invention specifically binds a polypeptide antigen. In another embodiment, a monomeric polypeptide of the invention specifically binds a nonpolypeptide antigen. In yet another embodiment, administration of a monovalent polypeptide of the invention to a mammal suffering from a disease or disorder can result in a therapeutic benefit in that mammal.

Virtually any molecule may be targeted by and/or incorporated into a monovalent polypeptide of the invention comprising a variant Fc variant portion (e.g., monovalent antibodies, Fc fusion proteins) including, but not limited to, the following list of proteins, as well as subunits, domains, motifs and epitopes belonging to the following list of proteins: renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VII, factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); hepatocyte growth factor (HGF); receptors for hormones or growth factors such as, for example, EGFR, VEGFR, HGFR (also known as cMET); interferons such as alpha interferon (α-IFN), beta interferon (β-IFN) and gamma interferon (γ-IFN); protein A or D; rheumatoid factors; a neurotrophic factor such as bone-derived neurotrophic factor (BDNF), neurotrophin-3,-4,-5, or -6 (NT-3, NT-4, NT-5, or NT-6), or a nerve growth factor; platelet-derived growth factor (PDGF); fibroblast growth factor such as αFGF and βFGF; epidermal growth factor (EGF); transforming growth factor (TGF) such as TGF-alpha and TGF-beta, including TGF-1, TGF-2, TGF-3, TGF-4, or TGF-5; insulin-like growth factor-I and-II (IGF-I and IGF-II); des (1-3)-IGF-I (brain IGF-I), insulin-like growth factor binding proteins; CD proteins such as CD2, CD3, CD4, CD 8, CD11a, CD14, CD18, CD19, CD20, CD22, CD23, CD25, CD33, CD34, CD40, CD40L, CD52, CD63, CD64, CD80 and CD147; TNF-related apoptosis-inducing ligand (TRAIL) receptors such as the death receptors TRAIL-R1 and TRAIL-R5 and the decoy receptors TRAIL-R3 and TRAIL-R5; erythropoietin; osteoinductive factors; immunotoxins; a bone morphogenetic protein (BMP); an interferon such as interferon-alpha,-beta, and-gamma; colony stimulating factors (CSFs), such as M-CSF, GM-CSF, and G-CSF; interleukins (ILs), e.g., IL-1 to IL-13; TNFα, superoxide dismutase; T-cell receptors; surface membrane proteins; decay accelerating factor; viral antigen such as, for example, a portion of the AIDS envelope, e.g., gp120; transport proteins; homing receptors; addressins; regulatory proteins; cell adhesion molecules such as LFA-1, Mac 1, p150.95, VLA-4, ICAM-1, ICAM-3 and VCAM, a4/p7 integrin, and (Xv/p3 integrin including either a or subunits thereof, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD 18, CD61, CD104, beta5, beta6, beta7 and beta8; Integrin subunit combinations including but not limited to, αVβ3, αVβ5 and αVβ7; Amyloid beta (Aβor Abeta); a member of an apoptosis pathway; blood group antigens; flk2/flt3 receptor; obesity (OB) receptor; mpl receptor; CTLA-4; protein C; an Eph receptor such as EphA2, EphA4, EphB2, etc.; a Human Leukocyte Antigen (HLA) such as HLA-DR; complement proteins such as complement receptor CR1, C1Rq and other complement factors such as C3, and C5; a glycoprotein receptor such as GpIba, GPIIb/IIIa and CD200; and fragments of any of the above-listed polypeptides.

Also contemplated are monovalent polypeptides of the invention that comprise an antigen binding portion that specifically bind cancer antigens including, but not limited to, ALK receptor (pleiotrophin receptor), pleiotrophin, KS 1/4 pan-carcinoma antigen; ovarian carcinoma antigen (CA125); prostatic acid phosphate; prostate specific antigen (PSA); melanoma-associated antigen p97; melanoma antigen gp75; high molecular weight melanoma antigen (HMW-MAA); prostate specific membrane antigen; carcinoembryonic antigen (CEA); polymorphic epithelial mucin antigen; human milk fat globule antigen; colorectal tumor-associated antigens such as: CEA, TAG-72, CO17-1A, GICA 19-9, CTA-1 and LEA; Burkitt's lymphoma antigen-38.13; CD19; human B-lymphoma antigen-CD20; CD33; melanoma specific antigens such as ganglioside GD2, ganglioside GD3, ganglioside GM2 and ganglioside GM3; tumor-specific transplantation type cell-surface antigen (TSTA); virally-induced tumor antigens including T-antigen, DNA tumor viruses and Envelope antigens of RNA tumor viruses; oncofetal antigen-alpha-fetoprotein such as CEA of colon, 5T4 oncofetal trophoblast glycoprotein and bladder tumor oncofetal antigen; differentiation antigen such as human lung carcinoma antigens L6 and L20; antigens of fibrosarcoma; human leukemia T cell antigen-Gp37; neoglycoprotein; sphingolipids; breast cancer antigens such as EGFR (Epidermal growth factor receptor); NY-BR-16; HER2 antigen (p185HER2); polymorphic epithelial mucin (PEM); malignant human lymphocyte antigen-APO-1; differentiation antigen such as I antigen found in fetal erythrocytes; primary endoderm I antigen found in adult erythrocytes; preimplantation embryos; I(Ma) found in gastric adenocarcinomas; M18, M39 found in breast epithelium; SSEA-1 found in myeloid cells; VEP8; VEP9; Myl; VIM-D5; D156-22 found in colorectal cancer; TRA-1-85 (blood group H); SCP-1 found in testis and ovarian cancer; C14 found in colonic adenocarcinoma; F3 found in lung adenocarcinoma; AH6 found in gastric cancer; Y hapten; Ley found in embryonal carcinoma cells; TL5 (blood group A); EGF receptor found in A431 cells; E1 series (blood group B) found in pancreatic cancer; FC10.2 found in embryonal carcinoma cells; gastric adenocarcinoma antigen; CO-514 (blood group Lea) found in Adenocarcinoma; NS-10 found in adenocarcinomas; CO-43 (blood group Leb); G49 found in EGF receptor of A431 cells; MH2 (blood group ALeb/Ley) found in colonic adenocarcinoma; 19.9 found in colon cancer; gastric cancer mucins; T5A7 found in myeloid cells; R24 found in melanoma; 4.2, GD3, D1.1, OFA-1, GM2, OFA-2, GD2, and M1:22:25:8 found in embryonal carcinoma cells and SSEA-3 and SSEA-4 found in 4 to 8-cell stage embryos; Cutaneous T-cell Lymphoma antigen; MART-1 antigen; Sialy Tn (STn) antigen; Colon cancer antigen NY-CO-45; Lung cancer antigen NY-LU-12 variant A; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; Tumor-Associated Antigen CO-029; Tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; and Cancer-Testis Antigen (NY-EOS-1); and fragments of any of the above-listed polypeptides.

In certain specific embodiments, a monovalent polypeptide of the invention comprising a variant Fc region (e.g., monovalent antibodies, Fc fusion proteins) comprises or binds to cMET or TRAIL-R2 or VEGF.

### 7.4 Monomeric Polypeptide Conjugates

In certain embodiments, the monomeric polypeptides of the invention are conjugated or covalently attached to a substance using methods well known in the art. In one embodiment, the attached substance is a therapeutic agent, a detectable label (also referred to herein as a reporter molecule) or a solid support. Suitable substances for attachment to monomeric polypeptides include, but are not limited to, an amino acid, a peptide, a protein, a polysaccharide, a nucleoside, a nucleotide, an oligonucleotide, a nucleic acid, a hapten, a drug, a hormone, a lipid, a lipid assembly, a synthetic polymer, a polymeric microparticle, a biological cell, a virus, a fluorophore, a chromophore, a dye, a toxin, an enzyme, a radioisotope, solid matrixes, semi-solid matrixes and combinations thereof. Methods for conjugation or covalently attaching another substance to a monomeric polypeptide are well known in the art.

In certain embodiments, the monomeric polypeptides of the invention are conjugated to a solid support. Monomeric polypeptides may be conjugated to a solid support as part of the screening and/or purification and/or manufacturing process. Alternatively monomeric polypeptides of the invention may be conjugated to a solid support as part of a diagnostic method or composition. A solid support suitable for use in the present invention is typically substantially insoluble in liquid phases. A large number of supports are available and are known to one of ordinary skill in the art. Thus, solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the monomeric polypeptides of the invention.

A suitable solid phase support can be selected on the basis of desired end use and suitability for various synthetic protocols. For example, where amide bond formation is desirable to attach the monomeric polypeptides of the invention to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TENTAGEL™, Rapp Polymere, Tubingen, Germany), polydimethyl-acrylamide resin (available from Milligen/Biosearch, California), or PEGA beads (obtained from Polymer Laboratories).

In certain embodiments, the monomeric polypeptides of the invention are conjugated to labels for purposes of diagnostics and other assays wherein the monomeric polypeptide and/or its associated ligand may be detected. A label conjugated to a monomeric polypeptide and used in the present methods and compositions described herein, is any chemical moiety, organic or inorganic, that exhibits an absorption maximum at wavelengths greater than 280 nm, and retains its spectral properties when covalently attached to a monomeric polypeptide. Labels include, without limitation, a chromophore, a fluorophore, a fluorescent protein, a phosphorescent dye, a tandem dye, a particle, a hapten, an enzyme and a radioisotope.

In certain embodiments, a monomeric polypeptide is conjugated to an enzymatic label. Enzymes are desirable labels because amplification of the detectable signal can be obtained resulting in increased assay sensitivity. Enzymes and their appropriate substrates that produce chemiluminescence are preferred for some assays. These include, but are not limited to, natural and recombinant forms of luciferases and aequorins.

In another embodiment, a monomeric polypeptide is conjugated to a hapten, such as biotin. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

In certain embodiments, a monomeric polypeptide is conjugated to a fluorescent protein label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents.

In certain embodiments, a monomeric polypeptide is conjugated to a radioactive isotope. Examples of suitable radioactive materials include, but are not limited to, iodine (¹²¹I, ¹²³K, ¹²⁵I, ¹³¹I), carbon (¹⁴C), (sulfur (³⁵S), tritium (³H), indium (¹¹¹In, ¹¹²In, ¹¹³mIn, ¹¹⁵mIn,), technetium (⁹⁹Tc, ⁹⁹ mTc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴⁰La, 175Yb, ¹⁶⁶Ho, ⁹⁰Y, ⁴⁷Sv, ¹⁸⁶Re, ¹⁸⁸Re, ¹⁴²Pr, ¹⁰⁵Rh and ⁹⁷Ru.

In certain embodiments, the monomeric polypeptides of the invention may be conjugated to a moiety that increases the pharmacokinetic properties of the polypeptide, such as a nonproteinaceous polymer or serum albumin. In one specific embodiment, the monomeric polypeptide is conjugated to a polymer, such as polyethylene glycol (“PEG”), polypropylene glycol, or polyoxyalkylenes, in the manner as set forth in U.S. Pat. No. 4,640,835; 4,496,689; 4,301,144; 4,670,417; 4,791,192 or 4,179,337. The term “PEG” is used broadly to encompass any polyethylene glycol molecule, without regard to size or to modification at an end of the PEG, and can be represented by the formula: X—O(CH₂CH₂O)ₙ₋₁CH₂CH₂OH (1), where n is 20 to 2300 and X is H or a terminal modification, e.g., a C₁₋₄ alkyl. In one embodiment, PEG may terminate on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. One skilled in the art can select a suitable molecular mass for PEG, e.g., based on how the pegylated binding polypeptide will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 10: 91-114 (1993).

PEG may be conjugated to a monomeric polypeptide of the invention using techniques known in the art. For example, PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap.21 and 22).

### 7.5 Nucleic Acids

In addition to the amino acid sequences described above, the invention further provides nucleotide sequences encoding the monomeric polypeptides of the invention that comprise a variant Fc region. Thus, the present invention also provides polynucleotide sequences encoding the monomeric polypeptides described herein as well as expression vectors containing such polynucleotide sequences for their efficient expression in cells (e.g., mammalian cells). The invention also provides host cells containing such polynucleotides and expression vectors as well as methods of making the monomeric polypeptides using the polynucleotides described herein. The foregoing polynucleotides encode monomeric polypeptides having the structural and/or functional features described herein.

The invention also encompasses polynucleotides that hybridize under stringent or lower stringency hybridization conditions, e.g., as defined herein, to polynucleotides that encode a monomeric polypeptide of the invention. The term “stringency” as used herein refers to experimental conditions (e.g., temperature and salt concentration) of a hybridization experiment to denote the degree of homology between the probe and the filter bound nucleic acid; the higher the stringency, the higher percent homology between the probe and filter bound nucleic acid.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 65° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides of the invention may be obtained, and the nucleotide sequence of the polynucleotides determined, by any method known in the art. For example, if the nucleotide sequence of all or a portion of the monomeric polypeptide is known, a polynucleotide encoding the polypeptide may be assembled from chemically synthesized oligonucleotides (e.g., as described in Kutmeier et al., BioTechniques 17:242 (1994)). Briefly, this involves synthesis of overlapping oligonucleotides containing portions of the sequence encoding the polypeptide, annealing and ligating of those oligonucleotides, and then amplifying the ligated oligonucleotides by PCR.

A polynucleotide encoding a monomeric polypeptide may also be generated from nucleic acid from a suitable source. If a clone containing a nucleic acid encoding a particular polypeptide is not available, but the sequence of the polypeptide molecule is known, a nucleic acid encoding the polypeptide may be chemically synthesized or obtained from a suitable source (e.g., a cDNA library, or a cDNA library generated from, or nucleic acid, preferably polyA+RNA, isolated from, any tissue or cells expressing the polypeptide by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide. Amplified nucleic acids generated by PCR may then be cloned into replicable cloning vectors using any method well known in the art.

Once the nucleotide sequence and corresponding amino acid sequence of the polypeptide is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY), to generate a polypeptide having a different amino acid sequence, for example to create amino acid substitutions, deletions, and/or insertions in an Fc region.

### 7.6 Vectors, Host Cells, And Polypeptide Production

Also provided herein are vectors that contain a polynucleotide encoding a monomeric polypeptide of the invention. In an exemplary embodiment, nucleic acids that encode a monomeric polypeptide as described herein may be incorporated into an expression vector in order to express the monomeric polypeptide in a suitable host cell. A variety of expression vectors may be utilized for monomeric polypeptide expression. Expression vectors may comprise self-replicating extra-chromosomal vectors or vectors which integrate into a host genome. Expression vectors are constructed to be compatible with the host cell type. Thus expression vectors, which find use in the present invention, include but are not limited to those which enable monomeric polypeptide expression in mammalian cells, bacteria, insect cells, yeast, and in vitro systems. As is known in the art, a variety of expression vectors are available, commercially or otherwise, that may find use for expressing monomeric polypeptides of the invention.

Expression vectors typically comprise a coding sequence for a monomeric polypeptide operably linked with control or regulatory sequences, selectable markers, and/or additional elements. By “operably linked” herein is meant that the nucleic acid coding for a monomeric polypeptide is placed into a functional relationship with another nucleic acid sequence. Generally, these expression vectors include transcriptional and translational regulatory nucleic acid operably linked to the nucleic acid encoding the monomeric polypeptide, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

The application also provides host cells comprising a nucleic acid, vector or expression vector that encode for a monomeric polypeptide and use of such host cells for expression of a monomeric polypeptide. Suitable host cells for expressing the polynucleotide in the vectors include prokaryotic, yeast, or higher eukaryotic cells. Suitable prokaryotes for this purpose include eubacteria, such as Gram-negative or Gram-positive organisms, for example, Enterobacteriaceae such as Escherichia coli. Eukaryotic microbes such as filamentous fungi or yeast are also suitable host cells, such as, for example, S. cerevisiae, Pichia, U.S. Pat. No. 7,326,681, etc. Suitable host cells for the expression of glycosylated polypeptides are derived from multicellular organisms, including plant cells (e.g., US20080066200), invertebrate cells, and vertebrate cells. Examples of invertebrate cells for expression of glycosylated monomeric polypeptides include insect cells, such as Sf21/5f9, Trichoplusia ni Bti-Tn5b1-4. Examples of useful vertebrate cells include chicken cells (e.g., WO2008142124) and mammalian cells, e.g., human, simian, canine, feline, bovine, equine, caprine, ovine, swine, or rodent, e.g., rabbit, rat, mink or mouse cells.

Mammalian cell lines available as hosts for expression of recombinant polypeptides are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the monomeric polypeptide. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any functional immunoglobulin chains), SP20, CRL7O3O and HsS78Bst cells. In one embodiment, human cell lines developed by immortalizing human lymphocytes can be used to recombinantly produce monomeric polypeptides. In one embodiment, the human cell line PER.C6. (Crucell, Netherlands) can be used to recombinantly produce monomeric polypeptides.

Also provided are methods for producing monomeric polypeptide utilizing the nucleic acids and host cells of the invention. Recombinant expression of a monomeric polypeptide generally requires construction of an expression vector containing a polynucleotide that encodes the monomeric polypeptide. The expression vector is then transferred to a host cell by conventional techniques, the transfected cells are then cultured by conventional techniques to produce a monomeric polypeptide. When expressing a monomeric antibody, the entire heavy and light chain sequences, including the variant Fc region, may be expressed from the same or different expression cassettes and may be contained on one or more vectors.

In certain embodiments, monomeric polypeptides of the invention are expressed in a cell line with stable expression of the monomeric polypeptide. Stable expression can be used for long-term, high-yield production of recombinant proteins. For example, cell lines which stably express the monomeric polypeptide molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Methods for producing stable cell lines with a high yield are well known in the art and reagents are generally available commercially.

In certain embodiments, monomeric polypeptides of the invention are expressed in a cell line with transient expression of the monomeric polypeptide. Transient transfection is a process in which the nucleic acid introduced into a cell does not integrate into the genome or chromosomal DNA of that cell. It is in fact maintained as an extrachromosomal element, e.g., as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art resulting in the expression and production of monomeric polypeptides. In certain embodiments, the mammalian cell culture media is based on commercially available media formulations, including, for example, DMEM or Ham's F12. In other embodiments, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The terms nutrient, ingredient, and component are used interchangeably herein to refer to the constituents that make up a cell culture medium.

Once a monomeric polypeptide molecule has been produced by recombinant expression, it may be purified by any method known in the art for purification of a polypeptide, for example, by chromatography (e.g., ion exchange, affinity, and sizing column chromatography), centrifugation, differential solubility, or by any other standard technique for the purification of proteins. Further, the monomeric polypeptides of the present invention may be fused to heterologous polypeptide sequences (such as “tags”) to facilitate purification. Examples of such tags include, for example, a poly-histidine tag, HA tag, c-myc tag, or FLAG tag. Antibodies that bind to such tag which can be used in an affinity purification process are commercially available.

When using recombinant techniques, the monomeric polypeptide can be produced intracellularly, in the periplasmic space, or directly secreted into the medium. If the monomeric polypeptide is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating polypeptides which are secreted into the periplasmic space of E. coli. Where the monomeric polypeptide is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

### 7.7 Pharmaceutical Formulations

In certain aspects the invention provides a pharmaceutical composition comprising a monomeric polypeptide according to the invention and a pharmaceutically acceptable excipient. In certain embodiments, at least 50%, 60%, 70%, 75%, 80%, 85%, 90%, 95%, 97%, 98%, 99% or 100% of the polypeptide comprising a variant Fc domain in the composition is monomeric. In certain embodiments, the percent of monomeric polypeptide is determined by SEC-MALLS. In certain embodiments, the percent of monomeric polypeptide is determined by AUC. In specific embodiments, the percent of monomeric polypeptide is determined by SEC-MALLS and/or AUC as described in the Examples set forth infra. In certain embodiments, the pharmaceutical composition of the invention is used as a medicament.

In certain embodiments, the monomeric polypeptides of the invention may be formulated with a pharmaceutically acceptable carrier, excipient or stabilizer, as pharmaceutical (therapeutic) compositions, and may be administered by a variety of methods known in the art. As will be appreciated by the skilled artisan, the route and/or mode of administration will vary depending upon the desired results. As used herein, the pharmaceutical formulations comprising the monomeric polypeptides are referred to as formulations of the disclosure. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. Other contemplated carriers, excipients, and/or additives, which may be utilized in the formulations of the invention include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of monomeric polypeptide, as well known those in the art or as described herein.

The formulations of the invention comprise a monomeric polypeptide in a concentration resulting in a w/v appropriate for a desired dose. In certain embodiments, the monomeric polypeptide is present in the formulation of the invention at a concentration of about 1 mg/ml to about 200 mg/ml, about 1 mg/ml to about 100 mg/ml, about 1 mg/ml to about 50 mg/ml, or 1 mg/ml and about 25 mg/ml. In certain embodiments, the concentration of the monomeric polypeptide in the formulation may vary from about 0.1 to about 100 weight %. In certain embodiments, the concentration of the monomeric polypeptide is in the range of 0.003 to 1.0 molar.

In one embodiment the formulations of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the invention should be sterile. The formulations of the invention may be sterilized by various sterilization methods, including sterile filtration, radiation, etc. In one embodiment, the monomeric polypeptide formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

Therapeutic compositions of the present invention can be formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Formulations of the present invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; US Publication No. 2004-0042972; and 2004-0042971).

The formulations may conveniently be presented in unit dosage form and may be prepared by any method known in the art of pharmacy. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages may range from about 0.0001 to about 100 mg/kg of body weight or greater, for example about 0.1, 1, 10, or 50 mg/kg of body weight, with about 1 to about 10 mg/kg of body weight being preferred.

### 7.8 Exemplary Uses

The monomeric polypeptides described herein may be used for diagnostic and/or therapeutic purposes. In certain embodiments, the monomeric polypeptides of the invention and compositions thereof may be used in vivo and/or in vitro for detecting target expression in cells and tissues or for imaging target expressing cells and tissues. For example, in certain embodiments, the monomeric polypeptides are monomeric antibodies comprising a variant Fc region that may be used to image target expression in a living human patient.

By way of example, diagnostic uses can be achieved, for example, by contacting a sample to be tested, optionally along with a control sample, with the monomeric antibody under conditions that allow for formation of a complex between the monomeric antibody and the target. Complex formation is then detected (e.g., using an ELISA or by imaging to detect a moiety attached to the monomeric antibody). When using a control sample along with the test sample, complex is detected in both samples and any statistically significant difference in the formation of complexes between the samples is indicative of the presence of the target in the test sample.

In one embodiment, the invention provides a method of determining the presence of the target in a sample suspected of containing the target, said method comprising exposing the sample to a monomeric antibody of the invention, and determining binding of the monomeric antibody to the target in the sample wherein binding of the monomeric antibody to the target in the sample is indicative of the presence of the target in the sample. In one embodiment, the sample is a biological sample.

In certain embodiments, the monomeric antibodies may be used to detect the overexpression or amplification of the target using an in vivo diagnostic assay. In one embodiment, the monomeric antibody is added to a sample wherein the monomeric antibody binds the target to be detected and is tagged with a detectable label (e.g., a radioactive isotope or a fluorescent label) and externally scanning the patient for localization of the label.

Alternatively, or additionally, FISH assays such as the INFORMT™ (sold by Ventana, Ariz.) or PATHVISIONT™ (Vysis, Ill.) may be carried out on formalin-fixed, paraffin-embedded tissue to determine the extent (if any) of the target expression or overexpression in a sample.

In certain aspects, the monomeric polypeptides and compositions thereof of the invention may be administered for prevention and/or treatment of a disease/disorder/condition in a subject in need thereof. The invention encompasses methods of preventing, treating, maintaining, ameliorating, or inhibiting a target associated or exacerbated disease/disorder/condition and/or preventing and/or alleviating one or more symptoms of the disease in a mammal, comprising administering a therapeutically effective amount of the monomeric polypeptide to the mammal. The monomeric polypeptide compositions can be administered short term (acute) or chronic, or intermittently as directed by physician.

### 8. EXEMPLIFICATION

The examples below are given so as to illustrate the practice of this invention. They are not intended to limit or define the entire scope of this invention.

### 8.1 Example 1

### Generation of Hinge-Deleted IgG4 Vector

The 12-amino acid hinge region of the wild-type human IgG4 constant domain was removed as follows: The IgG expression vector pEU8.2 has been derived from a heavy chain expression vector originally described in reference [1] and contains the human heavy chain constant domains and regulatory elements to express whole IgG heavy chain in mammalian cells. The vectors have been engineered simply by introducing an OriP element. An oligonucleotide primer was designed that flanked the 5′ intron upstream of the hinge region and the 3′ intron sequence directly downstream of the hinge region. Standard mutagenesis techniques as described in reference [2] were then employed to remove the upstream intron and 12 amino acid hinge region. The expected 420 bp deletion in the sequence was confirmed by DNA sequencing. The new vector was designated pEU8.2Δhinge.

### 8.2 Example 2

### Generation of Hinge-Deleted IgG4 Molecules

### 8.2.1 Example 2a

### Subcloning of Anti-cell-surface receptor Antibody 6 into pEU8.2Δhinge

V_{H} and V_{L} domains of an anti-cell surface receptor Antibody (designated “Antibody 6”) were subcloned into vectors pEU8.2Δhinge and pEU4.4 respectively. The V_{H} domain was cloned into a vector (pEU8.2Δhinge) containing the human heavy chain gamma 4 constant domains, but with the 12 amino acid hinge region removed, as well as regulatory elements to express whole IgG heavy chain in mammalian cells. Similarly, the V_{L} domain was cloned into a vector (pEU4.4) for the expression of the human light chain (lambda) constant domains and regulatory elements to express whole IgG light chain in mammalian cells. To obtain IgGs, the heavy and light chain IgG expressing vectors were transfected into EBNA-HEK293 mammalian cells. IgGs were expressed and secreted into the medium. Harvests were pooled and filtered prior to purification, then IgG was purified using Protein A chromatography. Culture supernatants were loaded on a column of appropriate size of Ceramic Protein A (BioSepra) and washed with 50 mM Tris-HCl pH 8.0, 250 mM NaCl. Bound IgG was eluted from the column using 0.1 M Sodium Citrate (pH 3.0) and neutralised by the addition of Tris-HCl (pH 9.0). The eluted material was buffer exchanged into PBS using Nap10 columns (Amersham, #17-0854-02) and the concentration of IgG was determined spectrophotometrically using an extinction coefficient based on the amino acid sequence of the IgG. The purified IgG were analysed for aggregation and degradation using SEC-HPLC and by SDS-PAGE.

### 8.2.2 Example 2b

### Characterisation of Antibody 6 IgG4Δhinge Molecules by Size Exchange. Chromatography coupled to Multi Angle Laser Light Scattering (SEC-MALLS)

Size Exclusion Chromatography coupled to Multi Angle Laser Light Scattering (SEC-MALLS) is a very sensitive technique for determining accurate molecular sizes of biopolymers. This system was used to determine the molecular weight of Antibody 6 IgG4Δhinge molecules compared to Antibody 6 IgG4 wild-type. 100 μl samples were firstly analysed using a BioSep-SEC-S 4000 column (300×7.8 mm, Phenomenex part number OOH-2147-K0, serial number 389524-11) which was equilibrated with Dulbecco's PBS at 1.0 mL min⁻¹ on an Agilent HP1100 HPLC. Peaks were detected using the 220 and 280 nm signals from a Diode Array Detector (DAD). Eluate from the HP1100 DAD detector was directed through Wyatt Technologies DAWN EOS and Optilab rEX detectors (Multiple Angle Light Scattering and Refractive Index detectors, respectively). The output of these detectors was processed using ASTRA V (5.1.9.1.) software. A refractive index increment (dn/dc) value of 0.184 was used (calculated assuming that glycosylated IgGs have ˜2.5% glycan by mass). The detector 11 (90°) background Light Scattering value from the D-PBS equilibrated columns was <0.35 Volts.

According to WO2007/059782 A1, the IgG4Δhinge variant should be approximately half of the size (˜75 kDa) of the wild-type IgG4 molecule. However, the calculated sizes for both the wild-type IgG4 and IgG4Δhinge were both around the expected size for a divalent molecule (Table 3). This indicates that the deletion of the 12 amino acid hinge region alone is not enough to produce a monovalent monomer of expected (˜75 kDa) size.

TABLE 3
Retention times and Calculated MW of Antibody 6 IgG4 and IgGΔhinge

Antibody 6 IgG4 Variant

| | IgG4 wild-type | IgG4Δhinge |
|---|---|---|
| Retention time BioSep-SEC | 9.394 | 9.465 |
| S 4000 (Minutes) | | |
| % Monomer Peak | >88 | >89 |
| % Multimer | 4.5 | 2.3 |
| MALS Mass (kDa) | 146 | 149 |

### 8.3 Example 3

### Generation of CH3 Constant Domain Mutations

In order to further stabilise the generation of monovalent antibodies, further mutations were introduced to the IgG4Δhinge molecule in the CH3 constant domain region to disrupt the CH3-CH3 interface between the two arms of the IgG4 molecule.

### 8.3.1 Example 3a

### Choice of Amino Acids for the Disruption of the CH3-CH3 Interface

The CH3 domain of IgG molecules contains the surface that promotes the dimerisation of two Fc chains to form the functional immunoglobulin molecule. Dimerisation is mediated by interactions within a single face on each of the two associating CH3 domains, the face on one CH3 domain being made up of identical amino acid residues to those in the face of the other CH3 domain and one of the CH3 domains being rotated 180° along its longitudinal axis relative to the other in order to achieve the correct orientation for dimerisation. The interface is made up of approximately 16 amino acids from each CH3 domain and, because of their relationship by rotational symmetry, the centre of the interface is made up of amino acids that are located at the same position in each of the protein chains. Analysis of the crystal structure [3] of the Fc domain of human IgG1 enabled the identification of threonine at position 366 and tyrosine at position 407 from both CH3 domains as being at the centre of the interface with each amino acid interacting with its counter part on the opposite CH3 domain. Alignment of the amino acid sequence of IgG1 CH3 domain with that of human IgG4 revealed the same amino acids were present in the sequence of IgG4, indeed the same amino acids are present at those positions in the CH3 domain of all human IgG isotypes. Substituting any of the amino acids in the CH3-CH3 interface could result in destabilisation of the interface and prevention of the formation of dimers, particularly if substitutions were made for amino acids with a larger side chain than the naturally occurring amino acid, as this would disrupt the intimate contacts necessary for strong interactions. Maximum disruption would be expected to be achieved by substituting an amino acid in one chain and the amino acid it contacted in the other chain. If the introduced amino acids carried the same net charge on their side chains this would be expected to produce charge based repulsion as well as disrupting the interacting surface through altered packing. In order to minimise the number of residues altered, the two amino acids at the centre of the interface were chosen, thr366 and tyr407, and were substituted with arginine, which has both a large side chain and carries a net positive charge.

### 8.3.2 Example 3b

### Mutagenesis of Antibody 6 IgG4Δhinge CH3 Domains

Standard site directed mutagenesis methods were used to mutate the threonine at position 366 to arginine and the tyrosine at position 407 to arginine of the pEU8.2Δhinge. The mutagenesis was confirmed using DNA sequencing. The new variant was designated pEU8.2ΔhingeT366RY407R. V_{H} and V_{L} domains of Antibody 6 were subcloned into vectors pEU8.2ΔhingeT366RY407R and pEU4.4 respectively. The V_{H} domain was cloned into a vector (pEU8.2ΔhingeT366RY407R) containing the human heavy chain gamma 4 constant domains, but with the 12 amino acid hinge region removed and the threonine at position 366 and tyrosine at position 407 mutated to arginine, as well as regulatory elements to express whole IgG heavy chain in mammalian cells. Similarly, the V_{L} domain was cloned into a vector (pEU4.4) for the expression of the human light chain (lambda) constant domains and regulatory elements to express whole IgG light chain in mammalian cells. To obtain IgGs, the heavy and light chain IgG expressing vectors were transfected into EBNA-HEK293 mammalian cells. IgGs were expressed and secreted into the medium. Harvests were pooled and filtered prior to purification, then IgG was purified using Protein A chromatography. Culture supernatants were loaded on a column of appropriate size of Ceramic Protein A (BioSepra) and washed with 50 mM Tris-HCl pH 8.0, 250 mM NaCl. Bound IgG was eluted from the column using 0.1 M Sodium Citrate (pH 3.0) and neutralised by the addition of Tris-HCl (pH 9.0). The eluted material was buffer exchanged into PBS using Nap10 columns (Amersham, #17-0854-02) and the concentration of IgG was determined spectrophotometrically using an extinction coefficient based on the amino acid sequence of the IgG. The purified IgG were analysed for aggregation and degradation using SEC-HPLC and by SDS-PAGE.

### 8.3.3 Example 3c

### Characterisation of Antibody 6 IgG4Δhinge T366RY407R molecules by Size Exchange Chromatography coupled to Multi Angle Laser Light Scattering (SEC-MALLS)

SEC-MALLS was used to determine the molecular weight of Antibody 6 IgG4Δhinge T366RY407R molecules compared to Antibody 6 IgG4 wild-type and Antibody 6 IgG4Δhinge. 100 μl samples were firstly analysed using a BioSep-SEC-S 4000 column (300×7.8 mm, Phenomenex part number 00H-2147-K0, serial number 389524-11) which was equilibrated with Dulbecco's PBS at 1.0 mL min⁻¹ on an Agilent HP1100 HPLC. Peaks were detected using the 220 and 280 nm signals from a Diode Array Detector (DAD). Eluate from the HP1100 DAD detector was directed through Wyatt Technologies DAWN EOS and Optilab rEX detectors (Multiple Angle Light Scattering and Refractive Index detectors, respectively). The output of these detectors was processed using ASTRA V (5.1.9.1.) software (Wyatt Technology Corporation, Santa Barbara, USA). A refractive index increment (dn/dc) value of 0.184 was used (calculated assuming that glycosylated IgGs have ˜2.5% glycan by mass). The detector 11 (90°) background Light Scattering value from the D-PBS equilibrated columns was <0.35 Volts.

The calculated size for the Antibody 6 IgG4Δhinge T366RY407R variant was approximately 68 kDa, consistent with a monovalent molecule, whereas both the wild-type IgG4 and IgG4Δhinge were both around the expected size for a divalent molecule (Table 4).

TABLE 4
Retention times and Calculated MW of Antibody 6 IgG4 Variants

| | Antibody 6 IgG4 Variants | | |
|---|---|---|---|
| | | | IgG4Δhinge |
| | IgG4 wild-type | IgG4Δhinge | T366RY407R |
| Retention time BioSep- | 9.394 | 9.465 | 9.841 |
| SEC S 4000 (Minutes) | | | |
| % Monomer Peak | >88 | >89 | >86 |
| % Multimer | 4.5 | 2.3 | 4.2 |
| MALS Mass (kDa) | 146 | 149 | 68 |

### 8.3.4 Example 3d

### Inhibition of Ligand-induced Cytokine Release from HeLa Cells

To determine the bioactivity of the monovalent Antibody 6 IgG4Δhinge T366RY407R compared to the bivalent Antibody 6 IgG4 wild-type and Antibody 6 IgG4Δhinge, their activity was evaluated in a HeLa human cell assay by measuring dose-dependent inhibition of ligand-induced cytokine release. Briefly, HeLa cells (European Collection of Cell Cultures, ECACC catalogue no. 93021013) maintained in MEM plus 10% fetal bovine serum plus 1% non-essential amino acids; were seeded in 96-well tissue culture assay plates at 1.5×10⁴ cells/well and cells were then cultured overnight (16-18 h) in a humidified atmosphere at 37° C. and 5% CO₂. The purified IgG variants serially diluted in culture media were added to the HeLa cells without removing overnight culture medium and pre-incubated with HeLa cells for 30-60 min at 37° C. This was followed by addition of an EC₅₀ concentration of ligand (defined as the concentration of ligand which gives a half maximal response in the assay) and incubation for 4-5 h in a humidified atmosphere at 37° C. and 5% CO₂. Supernatants (conditioned culture media) were harvested and cytokine levels in supernatants were determined using commercially available ELISA kits. The IC₅₀ for each construct tested is shown in Table 5. These data demonstrate that the monovalent Ab6 IgG4ΔhingeT366RY407R construct retains biological activity.

TABLE 5
IC50 Determinations

| | IC₅₀ in HeLa assay measuring | | |
|---|---|---|---|
| | ligand induced cytokine release | | |
| | (pM) | | |
| | N = 1 | N = 2 | N = 3 |
| Ab6 IgG4 | 53.8 | 61.1 | 98.7 |
| Ab6 IgG4Δhinge | 21.8 | 62.2 | 35.4 |
| Ab6 IgG4ΔhingeT366RY407R | 107 | 238 | 142 |
| Negative control clone CEA6 IgG4 | No effect | No effect | No effect |
| Negative control clone CEA6 | No effect | No effect | No effect |
| IgG4Δhinge | | | |

### 8.4 Example 4

### Molecular Modeling of the CH3-CH3 Interface

Analysis of the CH3-CH3 interface was performed with the high resolution crystal structure of a human IgG1 Fc domain (PDB accession number 1H3U [3] and the only available IgG4 Fc domain crystal structure (PDB accession number 1ADQ [4] using PyMol software (on the world wide web at pymol.org [5]]. The PDB accession numbers relate to the Protein Data Bank which can be assessed on the world wide web at pdb.org. Residues involved in intermolecular contacts were defined as those residues with any pair of atomic groups closer than the sum of their Van der Waal's radii plus 0.5 Å [6]. The potential disruptiveness of site-directed mutants was analysed using the PyMol mutagenesis wizard to identify theoretical clashes upon substitution with a different amino acid side chain.

Residues involved in intermolecular interactions at the CH3-CH3-interface are shown in Table 6. The most notable non-van der Waals interactions at the interface are two hydrogen bonds between T366 and Y407, which are present in all crystal structures analysed, and a possible three or four salt bridges (E356-K439, D399-R409, K392-D399, and R409-D399) depending on the structure.

T366 and Y407 are key residues at the core of the CH3 interface, with mutation of both of these residues to arginine preventing dimerisation of the Fc domain (see Example 3). A further two residues (L368 and F405) were identified as being involved in significant interactions in this region, suggesting that rational mutations at these locations may also prevent dimerisation of the CH3 domain. As stated previously, structural analysis showed the presence of up to 4 potential salt bridges at the dimerisation interface, with mutations at these positions that cause either a charge repulsion or simply remove electrostatic interaction predicted to have an impact on the formation of the Fc dimer. In addition to the four core interface residues (T366, L368, F405 and Y407) and the five salt bridge residues (E356, D399, K392, R409 and K439) a third set of five residues (L351, S364, L368, K370 T394) were identified as being opposite either the identical residue on the opposing CH3 domain of the homodimer or a specific residue that was deemed more likely to enable the insertion of a disruptive mutation (e.g., by insertion of like charges opposite each other). A fourth set of residues (Y349, S354, E357) on the periphery of the CH3-CH3 interface were also determined to be likely have an influence on dimer formation.

TABLE 6
The residues located at the CH3—CH3 interface in the crystal structure of
an IgG1 Fc domain (1H3U). Interface residues were determined by loss of
solvent accessibility and contact residues are those residues involved in
intermolecular contacts [6].

| | Interface | Contact |
|---|---|---|
| | Q347 | Q347 |
| | Y349 | Y349 |
| | T350 | T350 |
| | L351^{‡} | L351 |
| | P352^{‡} | |
| | S354 | S354 |
| | E356 | E356 |
| | E357 | E357 |
| | K360 | |
| | Q362 | |
| | S364 | S364 |
| | T366^{‡} | T366 |
| | L368 | L368 |
| | K370 | K370 |
| | N390 | |
| | K392 | K392 |
| | T393 | |
| | T394^{‡} | T394 |
| | P395^{‡} | P395 |
| | P396 | |
| | V397 | V397 |
| | L398 | L398 |
| | D399 | D399 |
| | S400 | |
| | F405 | F405 |
| | L406 | |
| | Y407^{‡} | Y407 |
| | S408 | |
| | K409 | K409 |
| | T411 | |
| | K439 | |
| No. of | 30 | 20 |
| Res | | |

^{‡}self-interacting residues

To analyse the influence of single or multiple site-directed mutations at these positions a set of five amino acids were chosen to be representative of each type of side chain: positive (arginine); negative (aspartate); large aromatic (tryptophan); small neutral (alanine); and hydrophilic (glutamine). Aliphatic side chains were avoided as it was reasoned that insertion of a hydrophobic group was not likely to disrupt a protein-protein interface. A total of 65 IgG4 CH3 domain single, double and triple mutants, shown in Table 7, were rationally designed and the constructs were expressed and analysed as hingeless IgG4 Fc domains. Of these mutants 21 were designed, expressed and analysed as IgG4 Fc domains with a wild type hinge and 37 IgG1 and 3 IgG2 hingeless Fc domain mutants were also investigated.

### 8.5 Example 5

### Mutagenesis of Amino Acids in CH3-CH3 Interface Region and Analysis by SEC-MALLS and HPLC

### 8.5.1 Example 5a

### Mutagenesis, Protein Expression and Purification

The CH2 and CH3 domains of IgG1, 2 and 4 were amplified by PCR from pre-existing antibody constructs and cloned into a pEU vector to generate expression constructs for hingeless Fc domains for the three IgG subclasses of interest. Oligonucleotide-directed mutagenesis was performed using the Stratagene QuikChange II Site-Directed Mutagenesis kit (Agilent Technologies, La Jolla, Calif., USA) according to the manufacturers' instructions.

Transient expression of recombinant Fc domains was performed in CHO cells transfected with the EBNA-1 gene. Cells containing 100 μg/ml Penicillin and Streptomycin were transfected at a cell count of 1±0.1×10⁶ viable cells/ml using linear PEI (polyethylenimine) at a PEI to DNA ratio of 12:1 with 1 μg of DNA per ml of cells. Cells were fed on days 2 and 5 with CHO CD Efficient Feed B (Invitrogen, Paisley, UK) and harvested by centrifugation after 7 days. The supernatant was filtered through a 0.22 μM filter and the Fc domains purified by protein G affinity chromatograph using Vivapure maxiprepG spin columns (Sartorius, Epsom, Surrey, UK). Eluted samples were concentrated and buffer exchanged into PBS using Nap10 columns (GE Healthcare, Uppsala, Sweden), with protein purity analysed by SDS-PAGE. Typical yields were approximately 50-100 mg of >95% pure protein per original litre of culture.

### 8.5.2 Example 5b

### Multi-Angle Laser Light Scattering

Light scattering was performed in-line with fractionation (SEC-MALLS), which was performed in the same manner as described above in Example 3b. Light scattering and differential refractive index were detected using the DAWN-HELEOS and Optilab rEX instruments respectively (Wyatt Technology Corp., Santa Barbara, Calif., USA). Data for mutants where available is shown in Table 7.

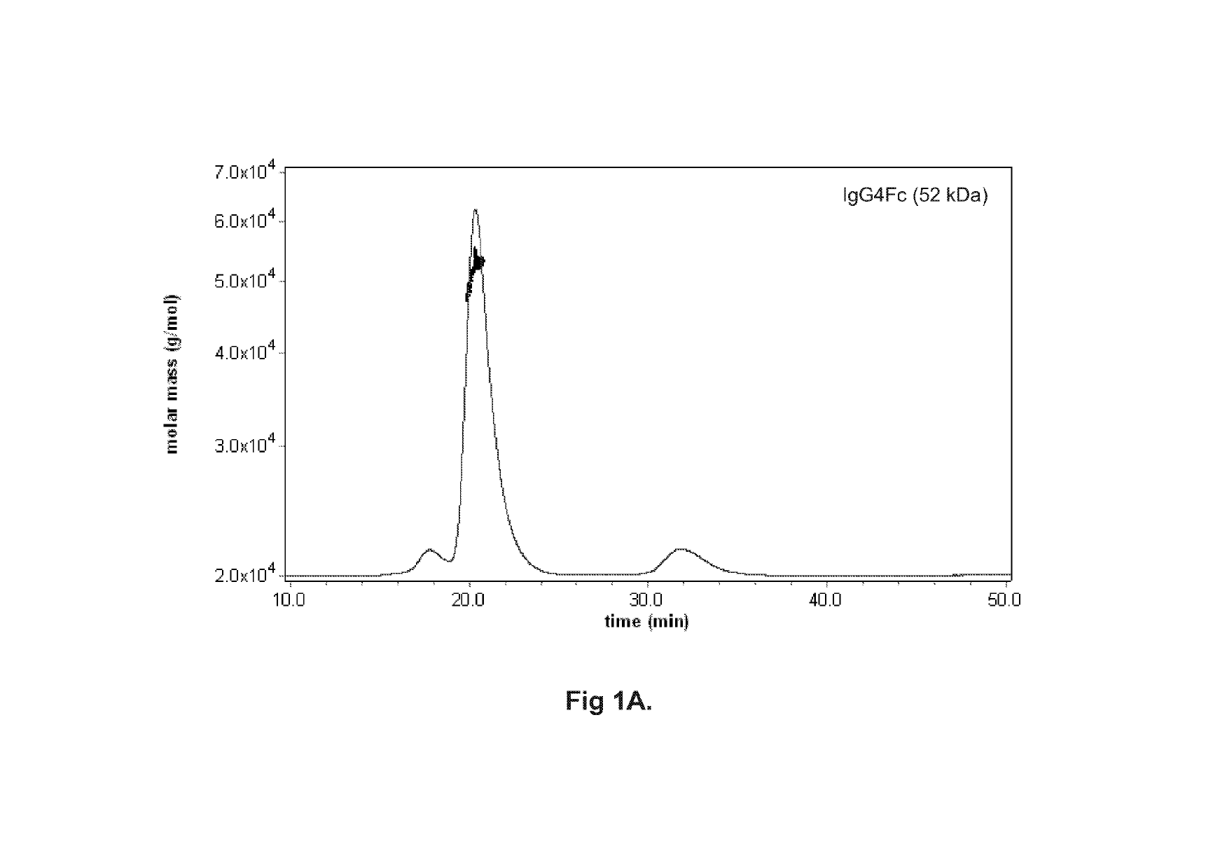

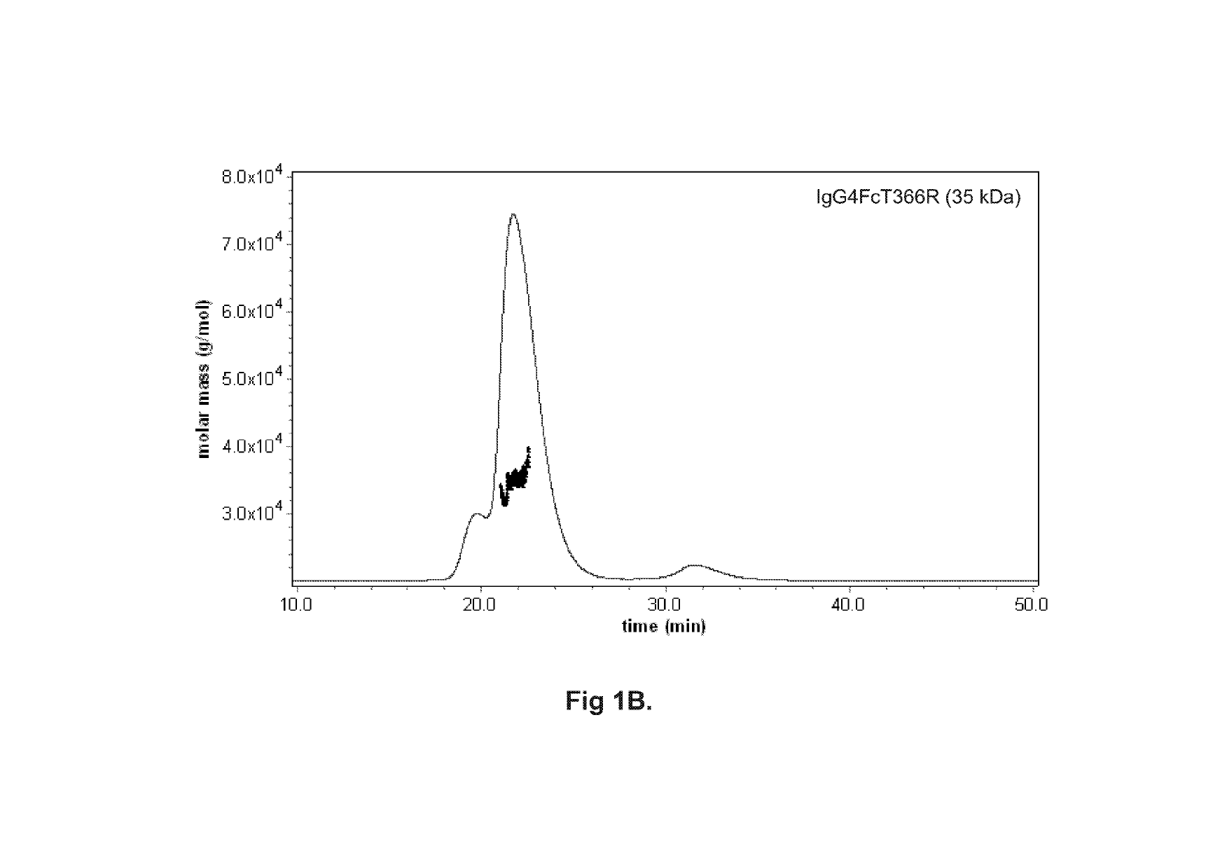

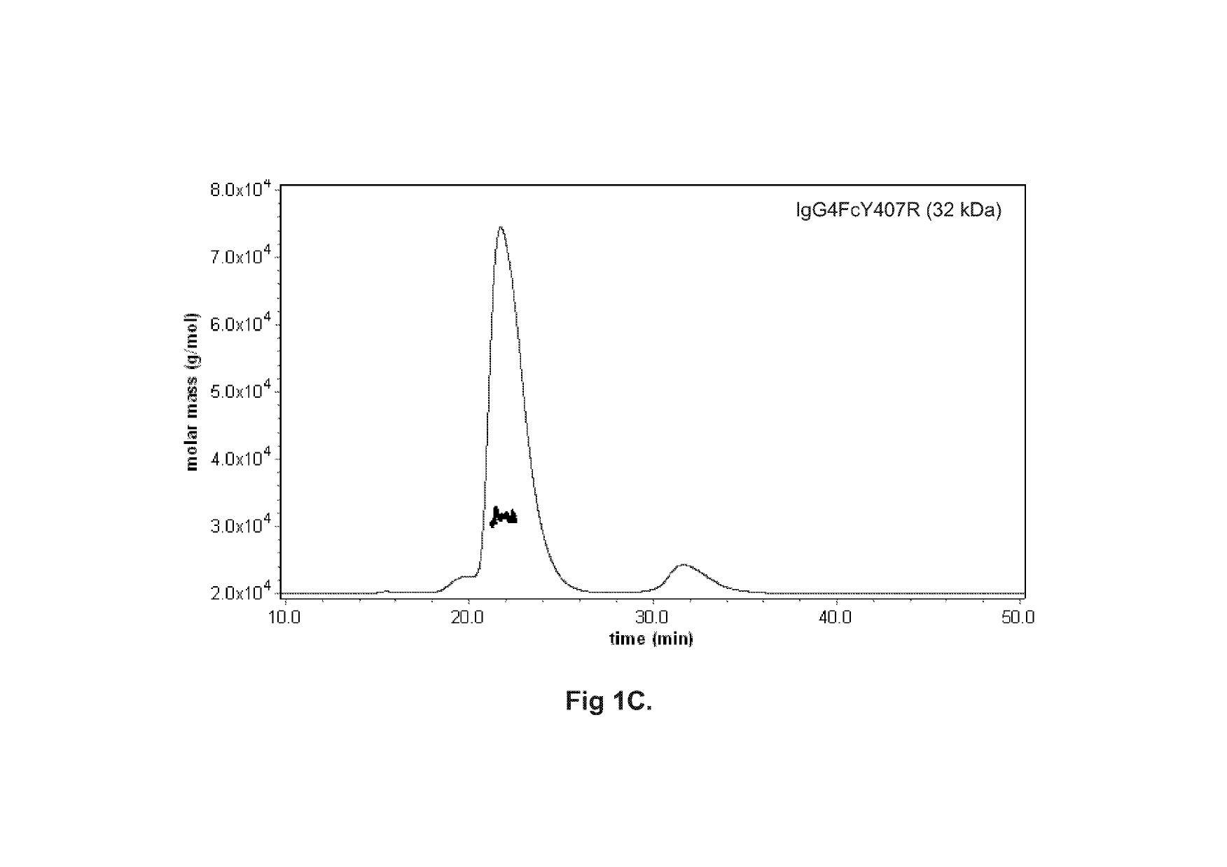

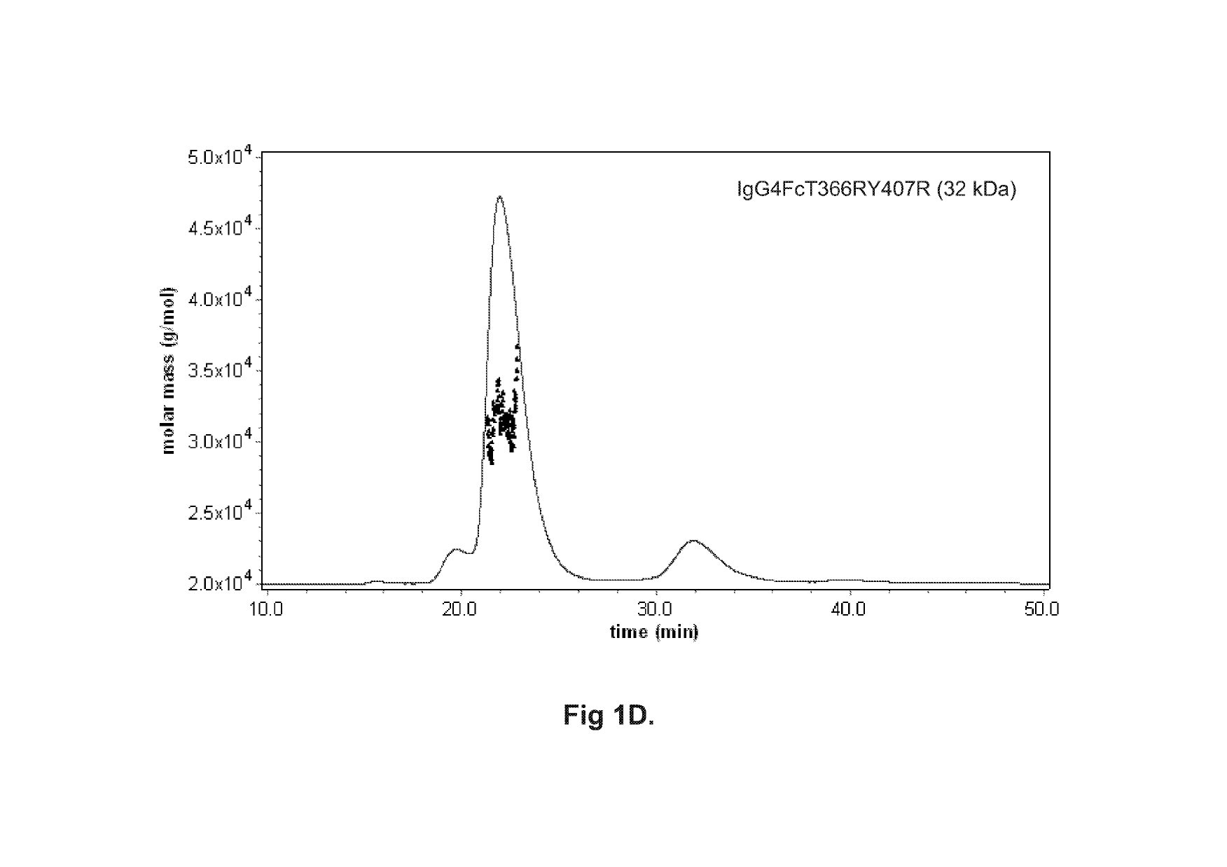

The Fc domains of the wild type IgG4 and T366R/Y407R double mutant, which had previously been analysed as full antibodies, were analysed by light scattering to determine an accurate measure (±3%) of the molecular weight of the protein and thus determine the monomeric or dimeric nature of the Fc domain. The single arginine mutants at positions 366 and 407 were also analysed as well as a further seven mutants. FIG. 1 shows the light scattering data for the T366R/Y407R samples compared to the wild type.

The molecular weight determined by MALDI-TOF mass spectrometry for the monomeric Fc domain was approximately 25.9 kDa (consisting of two equally populated glycoforms), with the dimer predicted to have a mass of 51.8 kDa. Therefore, the molecular weight of 52 kDa obtained from light scattering for the wild type IgG4 Fc domain corresponds well with the predicted molecular weight, suggesting that the wild type is exclusively dimeric under these conditions. However, the T366R, Y407R and T366R/Y407R mutants have lower apparent molecular weights (32-35 kDa), which are closer to but not completely consistent with that expected for a monomeric species.

### 8.5.3 Example 5c

### Size Exclusion Chromatography

Purified protein samples were analysed by size exclusion chromatography (SEC) using a Superdex 75 10/300 GL column (GE Healthcare, Uppsala, Sweden) on an Agilent 1100 series HPLC. 50 μl of each sample at a concentration of 0.8 mg/ml was injected onto the column using an autosampler with the run performed at a flow rate of 0.5 ml/min in Phosphate Buffered Saline running buffer. A sample of the wild type Fc domain was loaded with each batch for direct comparison and all samples were run in duplicate.

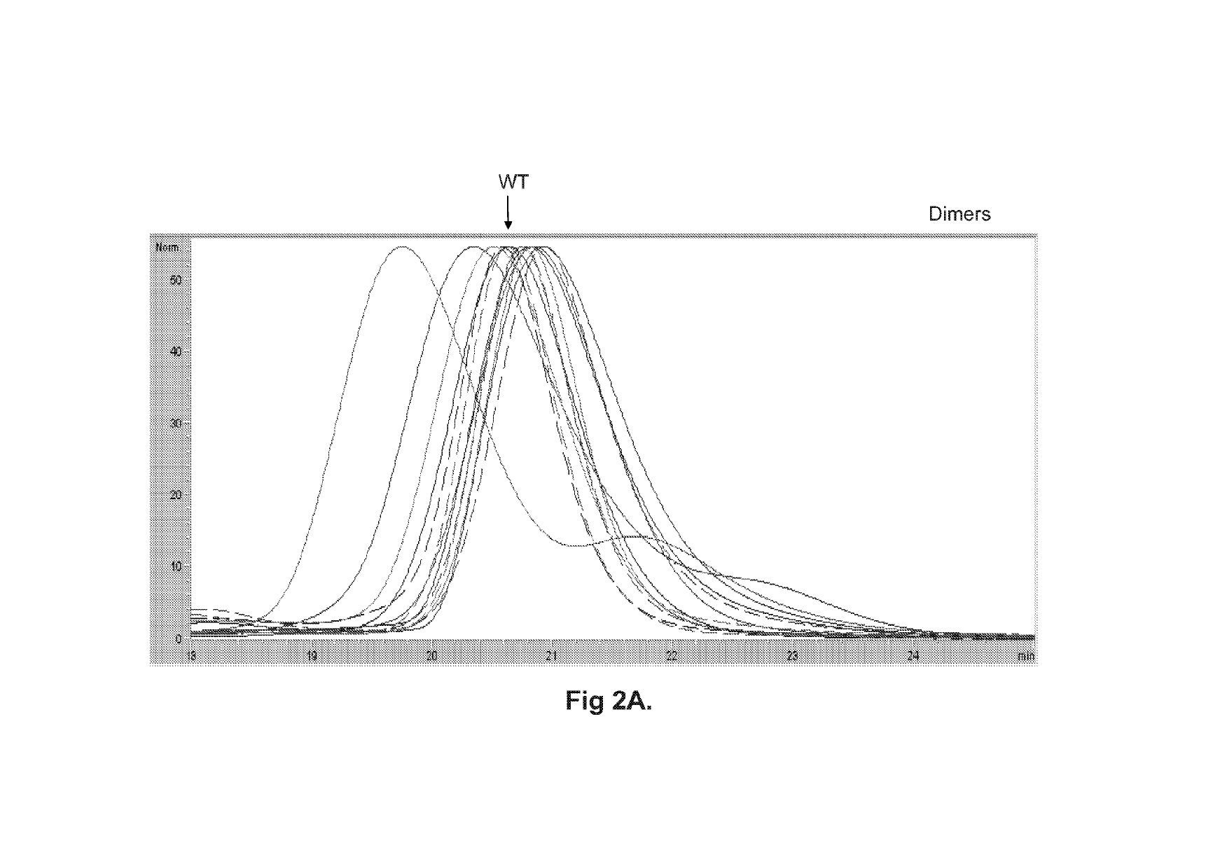

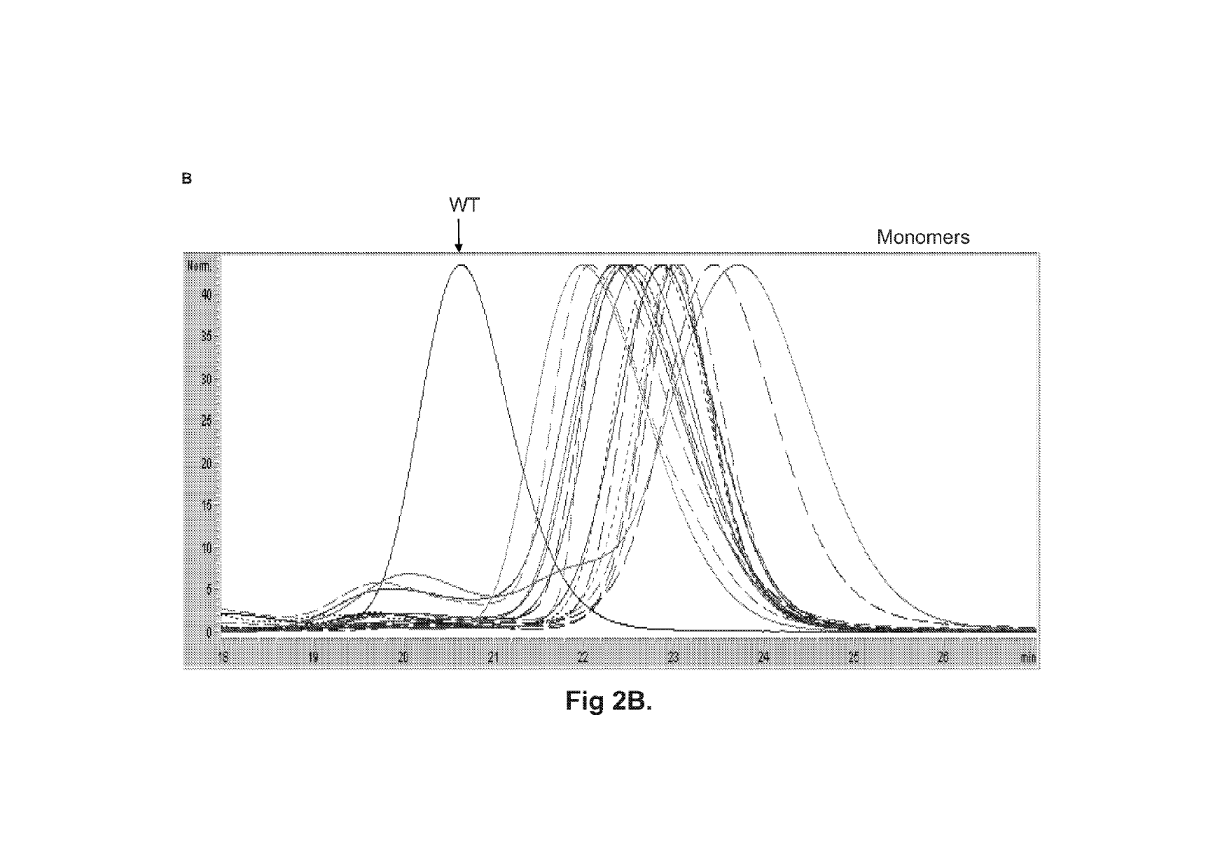

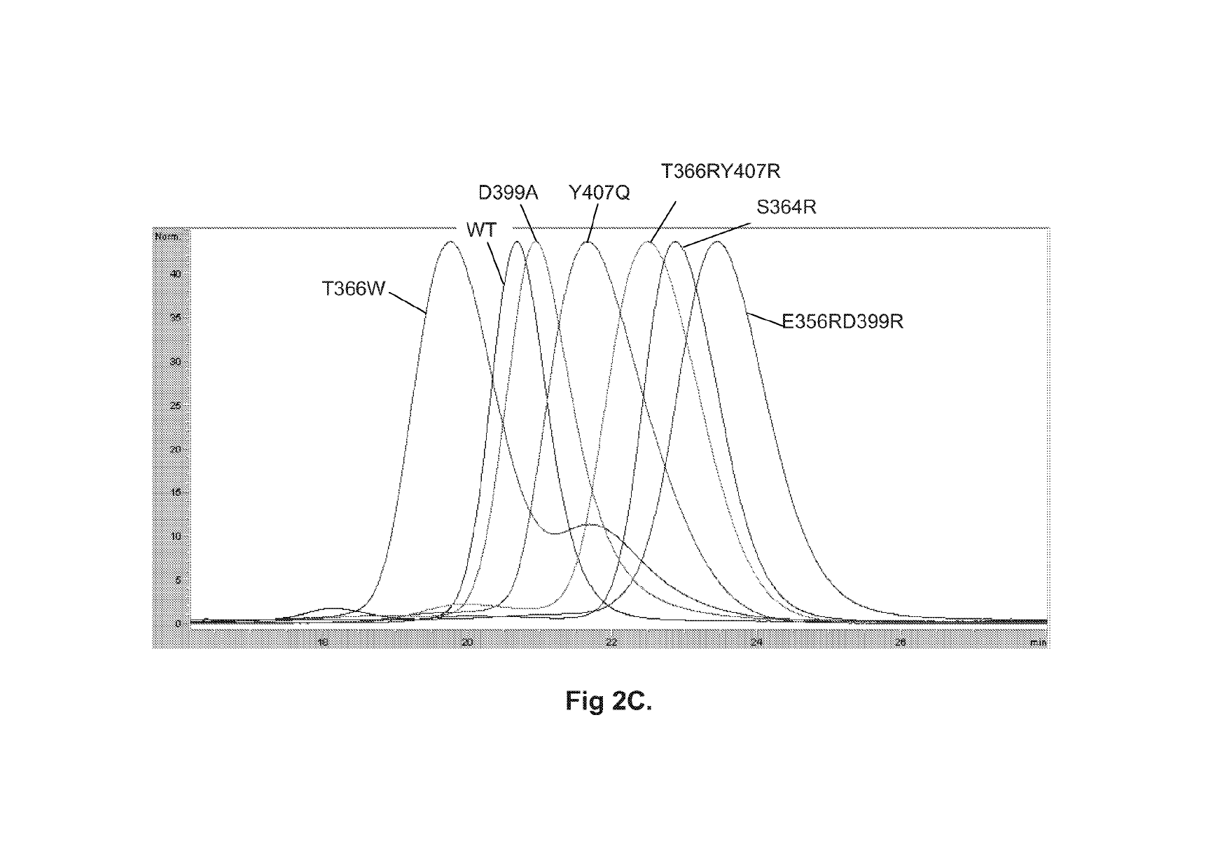

In agreement with the light scattering data, HPLC analysis of 65 IgG4 mutants revealed that the samples cannot be crudely separated into those that are dimers and those that are monomers, as FIG. 2 demonstrates. Table 7 shows data for 65 IgG4 mutants using size exclusion HPLC. Analysis revealed some IgG4 mutants which eluted with a molecular weight consistent with a dimer and other IgG4 mutants eluted with a molecular weight of a monomer. In addition, there were some IgG4 mutants which eluted with an intermediate retention time. It is believed that in these samples there is a rapid exchange between monomer and dimer the retention time being dependent on the equilibrium between these two species. Breaking down the mutants into three arbitrary groups based on SEC retention time and the appearance of the chromatogram (such as an apparently monodisperse sample, or an obvious mixture of species due to peak broadening or double peaks) results in 19 dimers (excluding the wild type), 18 in monomer-dimer equilibrium and 28 mutants that have a significantly smaller molecular weight indicative of a predominantly monomeric species. For the avoidance of doubt it would be clear to the skilled man that mutations which produce dimers when incorporated alone may lead to monomers when combined with other mutations which lead to monomers or species in equilibrium. Where the notation ‘monomer’ is used in the table the skilled man would be aware of further experimental techniques available to further investigate the structure of these species.

TABLE 7
A summary of the hingeless IgG4 mutants analysed by analytical size
exclusion using a Superdex 75 10/300 column at a flow rate of 0.5 ml/min.
The samples are ordered by retention time with calibration of the column
used to estimate molecular weight. The calculated molecular weight from
multi-angle laser light scattering (MALLS) is also shown for those
samples that the data is available for.

| Hingeless IgG4 | | | | MALLS |
|---|---|---|---|---|
| Fc Mutant | Analysis | RT (min) | SEC (kDa) | (kDa) |
| E356RK392DR409D | Dimer | 19.6 | 59.0 | |
| T366W | Dimer | 19.7 | 59.5 | 53 |
| T366D | Dimer | 20.3 | 54.0 | |
| K439D | Dimer | 20.5 | 52.5 | |
| K370W | Dimer | 20.5 | 52.5 | |
| K392AK439A | Dimer | 20.5 | 52.5 | |
| K439A | Dimer | 20.6 | 51.5 | |
| WT | Dimer | 20.6 | 51.5 | 52 |
| R409A | Dimer | 20.6 | 51.5 | |
| T366DY407D | Equilibrium | 20.7 | 51.0 | |
| D399W | Dimer | 20.7 | 51.0 | |
| S364W | Dimer | 20.7 | 51.0 | |
| S354D | Dimer | 20.7 | 51.0 | |
| K370A | Dimer | 20.7 | 51.0 | |
| E356AK392A | Dimer | 20.7 | 51.0 | |
| K392D | Dimer | 20.8 | 50.0 | |
| E356A | Dimer | 20.8 | 50.0 | |
| E356R | Dimer | 20.8 | 50.0 | |
| R409D | Dimer | 20.9 | 49.0 | |
| D399A | Dimer | 21.0 | 48.0 | |
| S354W | Dimer | 21.0 | 48.0 | |
| D399WR409W | Equilibrium | 21.0 | 48.0 | |
| D399AK439A | Equilibrium | 21.1 | 47.5 | |
| T366QY407Q | Equilibrium | 21.1 | 47.5 | |
| F405A | Equilibrium | 21.1 | 47.5 | 50 |
| E356RR409D | Equilibrium | 21.1 | 47.5 | |
| L351W | Equilibrium | 21.1 | 47.5 | |
| E356AD399AK439A | Equilibrium | 21.1 | 47.5 | |
| K392DK439D | Equilibrium | 21.2 | 46.5 | |
| Y349D | Equilibrium | 21.4 | 44.5 | 48 |
| L368W | Equilibrium | 21.5 | 44.0 | |
| Y407Q | Equilibrium | 21.6 | 43.5 | |
| T366Q | Equilibrium | 21.7 | 42.0 | |
| E356RK392D | Equilibrium | 21.8 | 41.5 | |
| Y407D | Monomer | 22.0 | 40.0 | |
| E356AD399A | Equilibrium | 22.0 | 40.0 | |
| T394W | Equilibrium | 22.0 | 40.0 | |
| Y407A | Equilibrium | 22.1 | 39.5 | |
| T394R | Monomer | 22.1 | 39.5 | |
| L351WT394W | Equilibrium | 22.2 | 38.5 | |
| T366R | Monomer | 22.3 | 37.5 | 35 |
| R409W | Monomer | 22.3 | 37.5 | |
| E357W | Monomer | 22.4 | 37.0 | |
| Y407R | Monomer | 22.4 | 37.0 | 32 |
| D399R | Monomer | 22.5 | 36.5 | |
| T366RY407R | Monomer | 22.5 | 36.5 | 32 |
| F405AY407A | Monomer | 22.6 | 36.0 | |
| Y349DS354D | Monomer | 22.6 | 36.0 | |
| T366QF405QY407Q | Monomer | 22.7 | 35.0 | |
| T394D | Monomer | 22.8 | 34.0 | 28 |
| F405Q | Monomer | 22.9 | 33.5 | |
| S364R | Monomer | 22.9 | 33.5 | |
| F405QY407Q | Monomer | 22.9 | 33.5 | |
| L351DT394D | Monomer | 23.0 | 33.0 | |
| L368R | Monomer | 23.0 | 33.0 | |
| L351D | Monomer | 23.0 | 33.0 | 29 |
| S364RL368R | Monomer | 23.1 | 32.0 | |
| L351R | Monomer | 23.1 | 32.0 | 30 |
| F405R | Monomer | 23.1 | 32.0 | 29 |
| L351RT394R | Monomer | 23.2 | 31.5 | |
| S364WL368W | Monomer | 23.3 | 31.0 | |
| E357R | Monomer | 23.4 | 30.0 | |
| D399RK439D | Monomer | 23.4 | 30.0 | |
| E356RD399R | Monomer | 23.4 | 30.0 | |
| T366WL368W | Monomer | 23.7 | 28.0 | |
| L351RS364RT394R | Monomer | 25.1 | 26.0 | |

To further investigate the role of the hinge region in Fc domain interactions seventeen of the monomeric hingeless IgG4 mutants as well as a small number of the other mutants were converted to IgG4 Fc domains with a wild type hinge and the purified proteins analysed by HPLC. All samples showed similar behaviour to that observed for the hingeless domains except for the R409W mutant, which contained almost equal populations of monomer and dimer compared to its behaviour as a predominantly monomeric species as a hingeless IgG4 Fc domain. The remaining 16 ‘monomeric’ mutants all contained less than 30% dimer as measured by peak integration (Table 8). This was shown to be a static population under non-reducing conditions as incubation at 37° C. for two weeks showed no clear signs of change by SDS-PAGE or HPLC. Table 9 summarizes the types of mutations that create monomeric Fcs (for IgG4 only) at the indicated positions.

TABLE 8
A table summarising the hinged IgG4 Fc mutants analysed by HPLC.
The mutants are ordered according to amount of dimer present in the
samples, with this being calculated by peak integration. The retention
time (RT) is used to estimate a molecular weight by comparison to a
calibration curve for the Superdex 75 10/300 column.

| Hinged IgG4 | | RT | SEC | % |
|---|---|---|---|---|
| Fc Mutant | Analysis | (min) | (kDa) | dimer |
| T366W | Dimer | 19.5 | 59.5 | 100.0 |
| Wild type | Dimer | 20.1 | 56.5 | 100.0 |
| S364W | Dimer | 20.1 | 56.5 | 100.0 |
| F405A | Dimer | 20.2 | 56.0 | 100.0 |
| T366Q | Equilibrium | 20.2 | 56.0 | 58.3 |
| R409W | Equilibrium | 20.1 | 56.5 | 56.4 |
| D399R | Monomer | 22.2 | 38.5 | 26.8 |
| L351D | Monomer | 22.5 | 36.5 | 23.0 |
| L351R | Monomer | 22.6 | 36.0 | 20.9 |
| L351DT394D | Monomer | 22.0 | 40.0 | 20.6 |
| F405Q | Monomer | 22.5 | 36.5 | 18.0 |
| S364WL368W | Monomer | 22.9 | 33.5 | 16.5 |
| L368R | Monomer | 22.5 | 36.5 | 12.4 |
| F405R | Monomer | 22.6 | 36.0 | 6.2 |
| L351RT394R | Monomer | 22.7 | 35.5 | 5.8 |
| T366R | Monomer | 22.0 | 40.0 | 5.4 |
| T366RY407R | Monomer | 22.5 | 36.5 | 5.1 |
| T394D | Monomer | 22.3 | 37.5 | 5.0 |
| T366WL368W | Monomer | 23.7 | 28.0 | 3.7 |
| S364R | Monomer | 22.4 | 37.0 | 3.2 |
| Y407R | Monomer | 22.0 | 40.0 | 2.3 |
| S364RL368R | Monomer | 22.6 | 36.0 | 1.5 |

TABLE 9
A representation of the type and position of single mutations that
lead to the formation of a monomeric-Fc domain. Mutations resulting
in a monomeric Fc are represented by a tick (✓) and mutants
that do not form monomeric Fcs are indicated by a cross (x).

| | Positive | Negative | Large | Small | Hydrophilic |
|---|---|---|---|---|---|
| Y349 | | x | | | |
| L351 | ✓ | ✓ | x | | |
| S354 | | x | x | | |
| E356 | x | | | x | |
| E357 | ✓ | | ✓ | | |
| S364 | ✓ | | x | | |
| T366 | ✓ | x | x | | x |
| L368 | ✓ | | x | | |
| K370 | | | x | x | |
| K392 | | x | | | |
| T394 | ✓ | ✓ | x | | |
| D399 | ✓ | | x | x | |
| F405 | ✓ | | | x | ✓ |
| Y407 | ✓ | ✓ | | x | x |
| R409 | | x | ✓ | x | |
| K439 | | x | | x | |

### 8.6 Example 6

### HPLC Analysis of IgG1 and 2

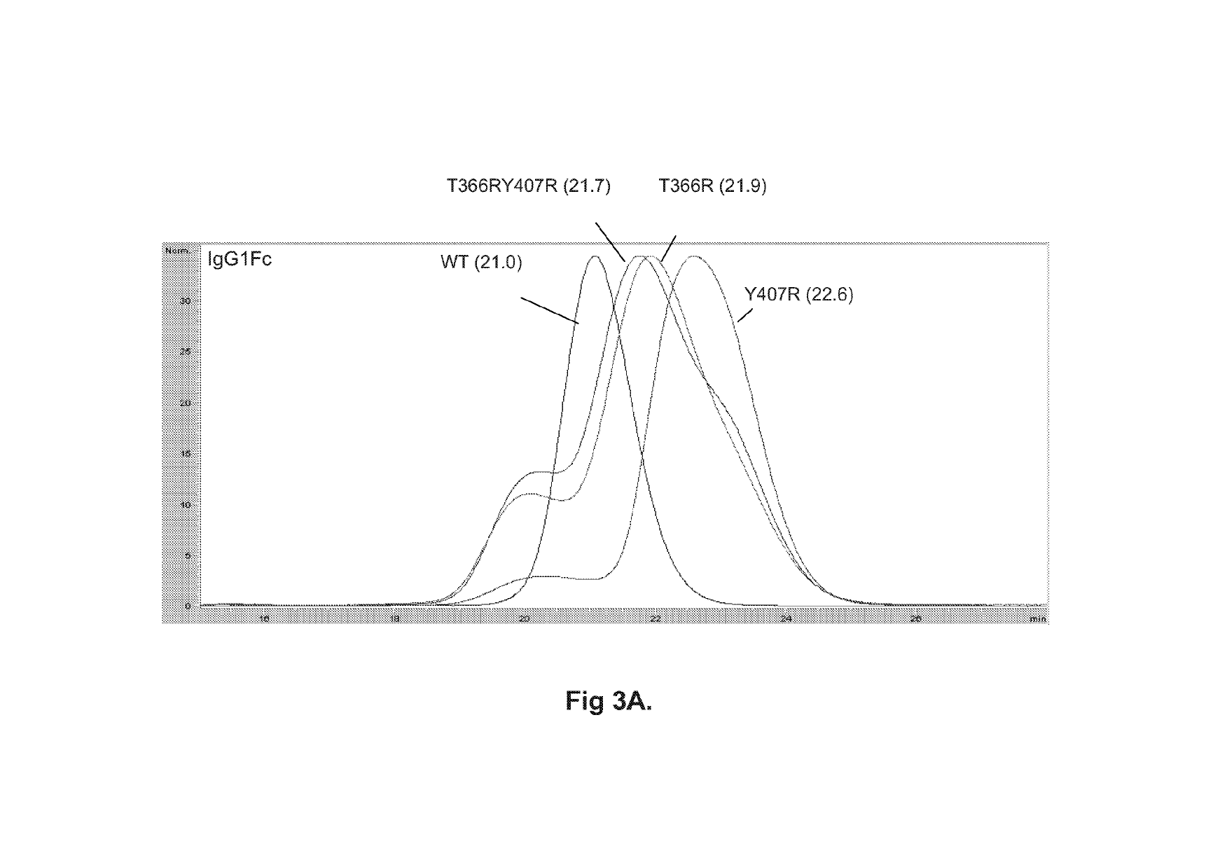

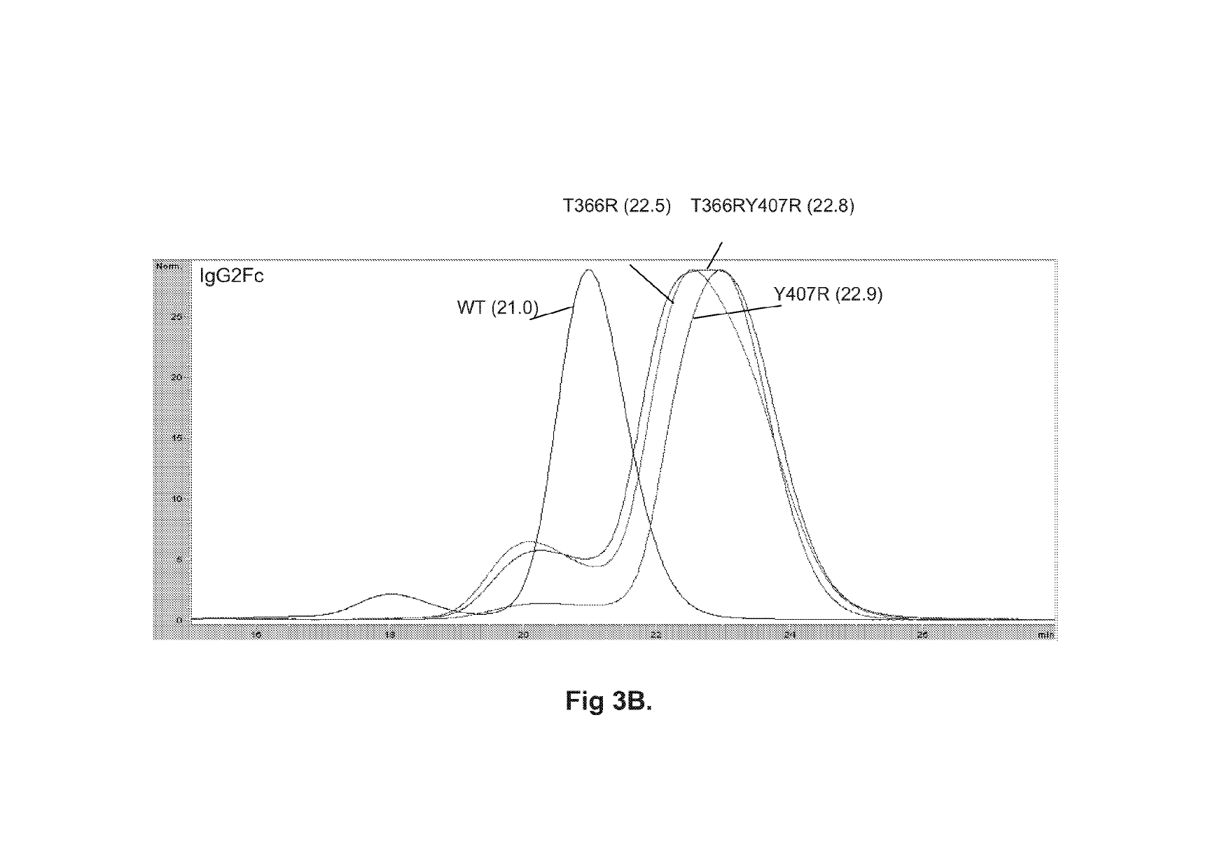

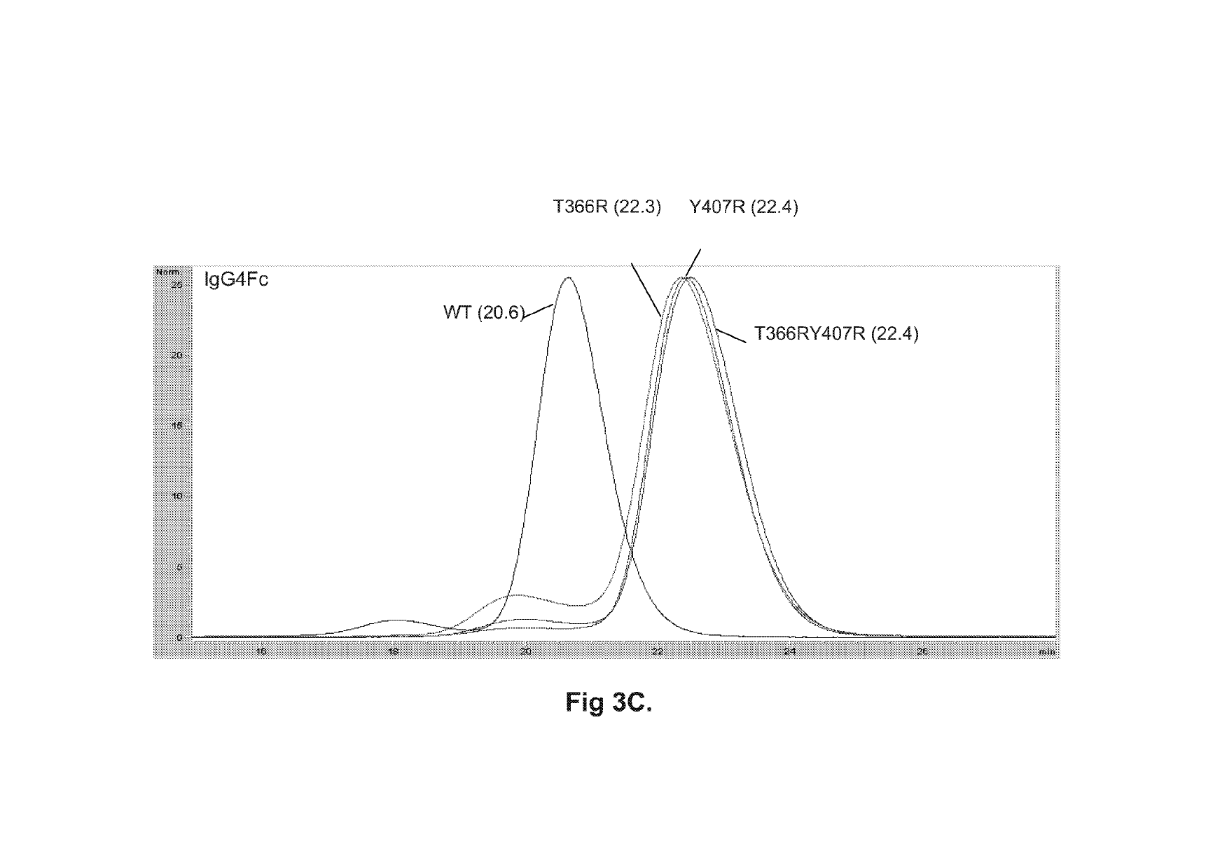

The chromatograms in FIG. 3 show the analytical SEC data for the single and double T366R/Y407R mutants for IgG subclasses 1 and 2 compared to those for IgG4. The mutants of the three subclasses behave differently, despite having almost identical interface residues by sequence alignment. For both IgG1 and 2 the Y407R mutant appears to be the most monomeric in nature, with the T366R and T366R/Y407R mutants showing clear signs of a mixed population. This was analysed further by generation of 29 hingeless IgG1 Fc domain mutants. Of the 21 mutants investigated that were monomeric as the IgG4 subtype only 11 were monomeric as IgG1 (Table 10).

Three of the residues which differ between the IgG subclasses, R355Q, Q419E and P445L, are not involved in intermolecular interactions and so should have no major influence on the stability of the CH3 dimer. However, R409K is at the interface between the two CH3 domains and K409 has previously been shown to contribute heavily to the stability of the Fc dimer [7]. Site-directed mutagenesis of the IgG1 mutants to produce an IgG4-like interface (i.e., K409R) resulted in some of the mutants reverting to the state observed for IgG4, as evident in Table 10.

This work represents the first engineering and characterisation of stable half-antibodies, which provides a solution to the sometimes undesired agonistic affects that cross-linking of antigens by bivalent antibodies can have while maintaining the advantageous properties of the Fc domain, such as prolonged half-life. This is a unique property that other non-activating antibody formats or novel scaffolds do not posses without fusion to a peptide, protein or polymer that extends half-life via increased size and/or FcRn recycling, thus making the monovalent antibody an attractive alternative.

TABLE 10
An overview of the monomeric mutants for hingeless IgG4 Fc, hinged
IgG4 Fc and hingeless IgG1 Fc domains. A monomeric, as determined by
HPLC, is represented by a tick (✓), with mutants that are dimeric or in
monomer-dimer equilibrium represented by a cross (x) and mutants for
which there is no data are left blank.

| | | | | Hingeless |
|---|---|---|---|---|
| | Hingeless | Hinged | Hingeless | IgG1 Fc |
| Mutant | IgG4 Fc | IgG4 Fc | IgG1 Fc | K409R |
| L351D | ✓ | ✓ | x | ✓ |
| L351R | ✓ | ✓ | x | |
| E357R | ✓ | | | |
| E357W | ✓ | | | |
| S364R | ✓ | ✓ | x | ✓ |
| T366R | ✓ | ✓ | x | x |
| L368R | ✓ | ✓ | ✓ | ✓ |
| T394D | ✓ | ✓ | ✓ | ✓ |
| T394R | ✓ | | x | |
| D399R | ✓ | ✓ | x | ✓ |
| F405Q | ✓ | ✓ | ✓ | |
| F405R | ✓ | ✓ | | |
| Y407D | ✓ | | x | x |
| Y407R | ✓ | ✓ | ✓ | |
| R409W | ✓ | x | x | |
| Y349DS354D | ✓ | | | |
| L351DT394D | ✓ | ✓ | ✓ | |
| L351RT394R | ✓ | ✓ | ✓ | |
| E356RD399R | ✓ | | ✓ | |
| S364RL368R | ✓ | ✓ | ✓ | |
| S364WL368W | ✓ | ✓ | ✓ | |
| T366RY407R | ✓ | ✓ | x | x |
| T366WL368W | ✓ | ✓ | ✓ | |
| D399RK439D | ✓ | | x | |
| F405AY407A | ✓ | | | |
| F405QY407Q | ✓ | | | |
| L351RS364RT394R | ✓ | | ✓ | |
| T366QF405QY407Q | ✓ | | | |

### 8.7 Example 7

### Sedimentation Velocity Analytical UltraCentrifugation (SV-AUC)

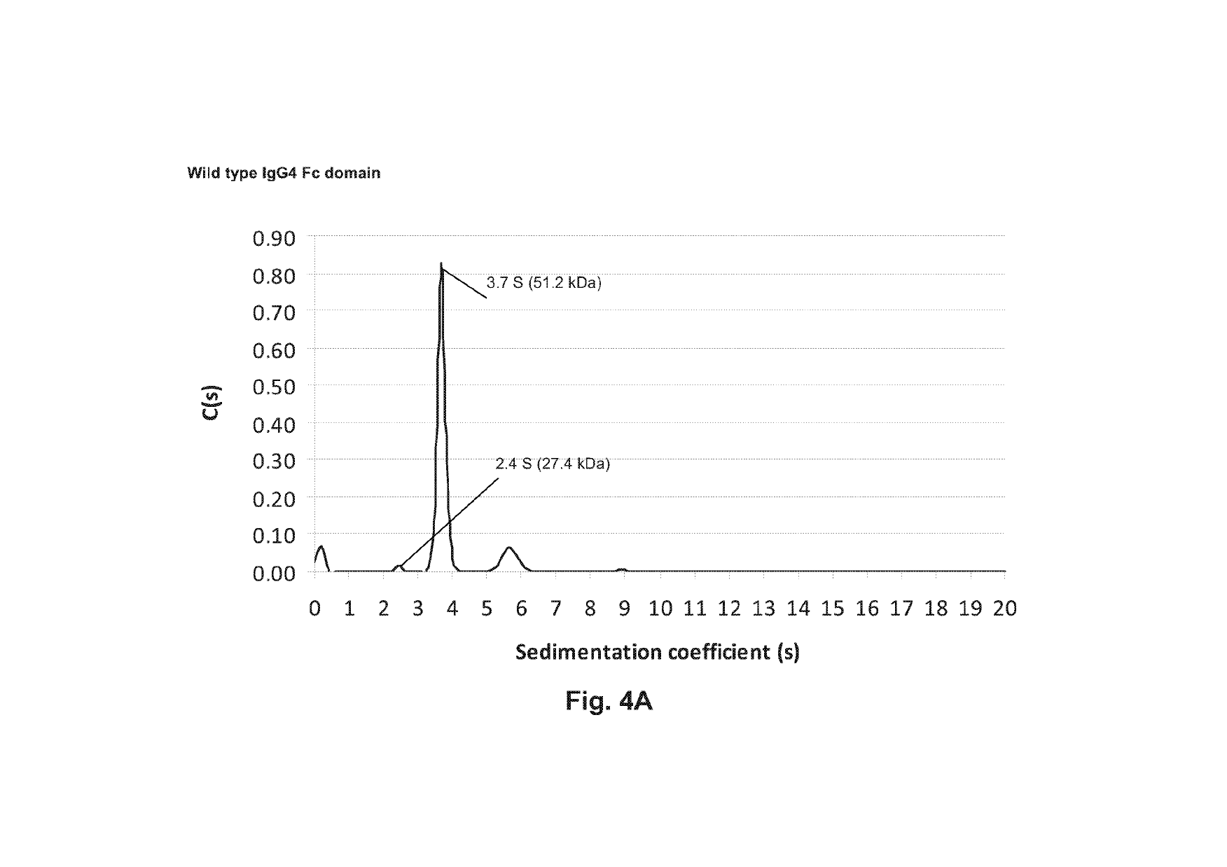

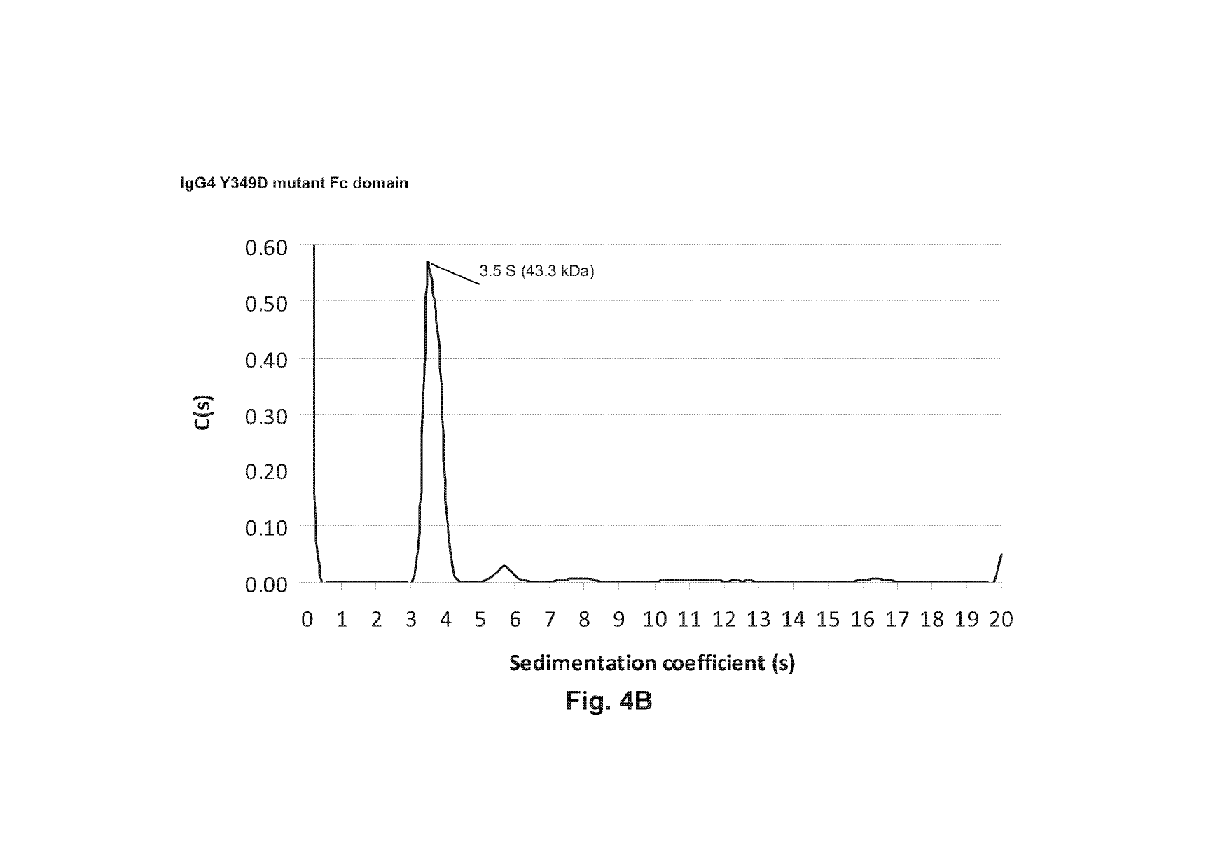

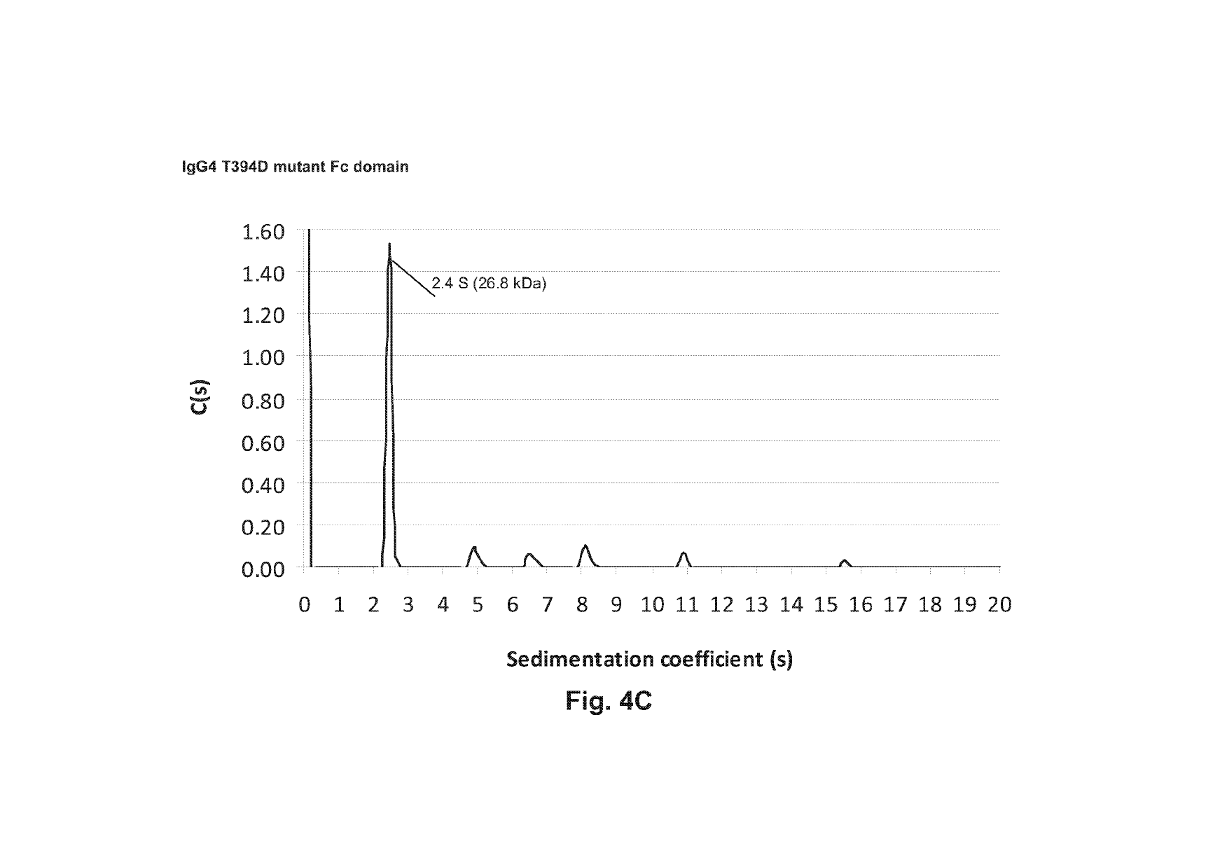

Sedimentation Velocity Analytical UltraCentrifugation (SV-AUC) was performed on several hingeless constructs to determine the sedimentation coeffiecients and the apparent in solution molecular weight. Experiments and analysis was performed at M-Scan Ltd. (Wokingham, UK). SV-AUC was undertaken on a Beckman Coulter XL-A AUC instrument at 20° C. Samples at concentrations between 28 and 42 μM were loaded into the sample sectors of the XL-A AUC cells with PBS buffer in the reference sector of the cells. A wavelength (λ) scan was performed to obtain a suitable λ that could be used for the subsequent scans (where the data obtained was in a spectral region where the Beer Lambert law remained valid i.e. with an absorbance of <1.0). The λ of 300 nm was chosen on this basis. Initial SV scans were undertaken at 3,000 rpm to check for the presence of heavy aggregates. No boundary movements were observed indicating the absence of large precipitates in the samples. A final rotor speed of 40,000 rpm was selected with 200 scans at 6 minute intervals. The data obtained was assessed using the SEDFIT program to obtain the c(s) profile of the sedimentation coefficient (s) values, reported in Svedberg units (S). An average partial specific volume of 0.73 ml/g (at 20° C.) was used in the SEDFIT analysis. The computer program SEDNTERP was used to calculate the buffer density and viscosity of PBS. A buffer density value of 1.00534 and buffer viscosity (Poise) of 0.01002 was calculated. A summary of the sedimentation coefficients obtained for three hingeless Fc samples is shown in Table 11. The distribution graphs of this data are represented in FIG. 4.

The major species for the wild type hingeless IgG4 Fc domain gave an s value of 3.7 S. A conversion to c(M) gave the 3.7 S component an apparent in solution molecular weight of 51.2 kDa, which is in agreement with the expected molecular mass of the homodimer. A smaller component with an s value of 2.4 S and relative percentage UV absorbance of 1.2% has an apparent in solution molecular weight of 27.4 kDa, which is in close agreement to the expected mass of the monomer (FIG. 4A). The major species for the hingeless IgG4 Y349D Fc domain gave an s value of 3.5 S. Conversion to c(M) gave the 3.5 S component an apparent molecular weight of 43.3 kDa, which is lower than expected for the homodimer component. This conclusion agrees with HPLC data suggesting that this particular mutant is in rapid-monomer-dimer equilibrium (FIG. 4B). The major species for the hingeless IgG4 T394D Fc domain gave an s value of 2.4 S. Conversion to c(M) gave the 2.4 S component an apparent molecular weight of 26.8 kDa, which is in agreement with the expected molecular mass of the monomeric Fc domain. The presence of homodimer was not detected for this mutant (FIG. 4C).

TABLE 11
Summary of the sedimentation coefficients determined by
SV-AUC and calculated molecular weight of the major
species for three hingeless IgG4 Fc domains.

| | | Mol. Wt. of |
|---|---|---|
| | | major species |
| Sample | Sed. coef. values (S) | (kDa) |
| WT hingeless IgG4 Fc | 2.4, 3.7, 5.7, 8.9 | 51.2 |
| domain | | |
| hingeless IgG4 Y349D Fc | 3.5, 5.7, 7.9, 10.9, 16.4 | 43.3 |
| domain | | |
| hingeless IgG4 T394D Fc | 2.4, 4.9, 6.5, 9.1, 10.9, | 26.8 |
| domain | 15.6 | |

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### 8.8 Example 8

### Pharmacokinetic Studies in Mice

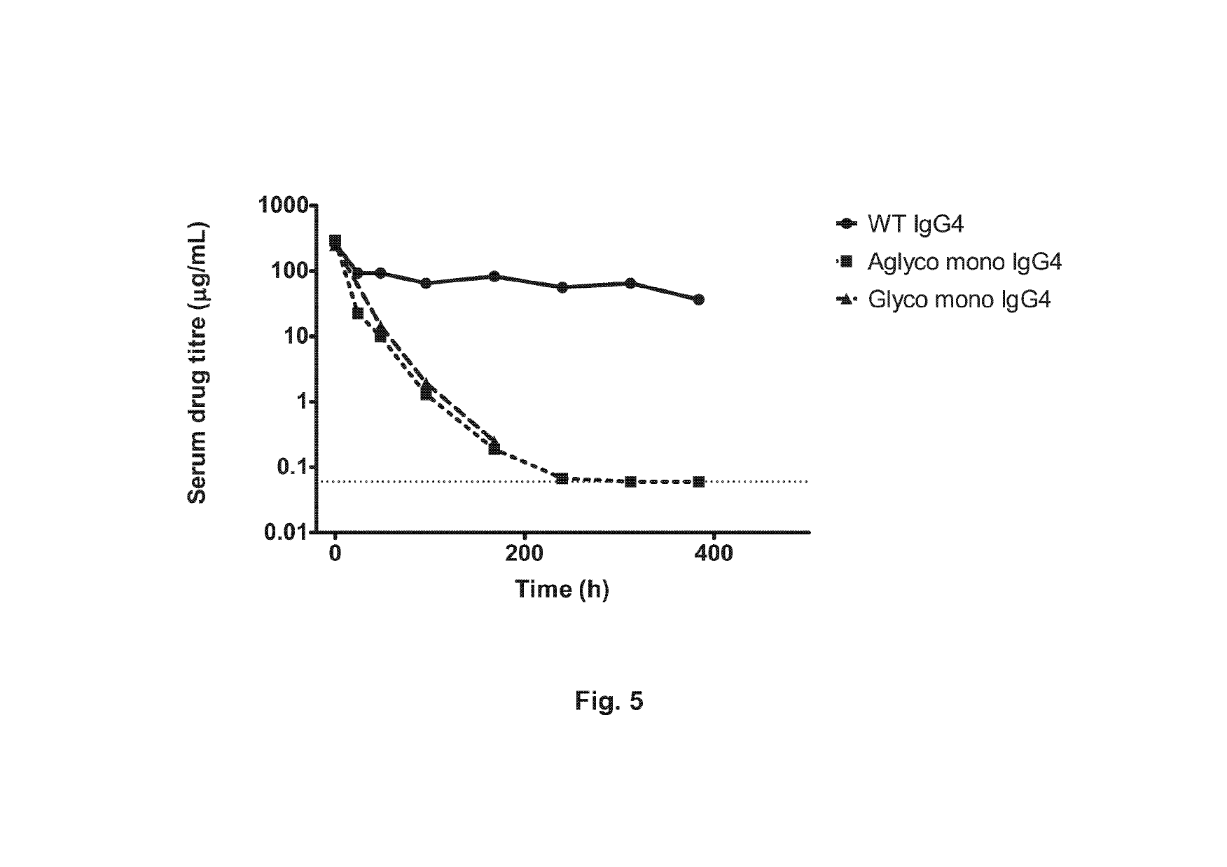

BALB/c mice were given a 10 mg/kg body weight IV bolus dose of a wild type IgG4, glycosylated monovalent IgG4 (consisting of C226Q/C229Q/T394D mutations) or an aglycosylated monovalent IgG4 (consisting of C226Q/C229Q/N297Q/T394D mutations) with 5 mice per group. Plasma samples were collected at 5 minutes, 1, 2, 4, 7, 10, 13 and 16 days for the wild type IgG4 and aglycosylated monovalent IgG4 and at 5 minutes, 2, 4 and 7 days for the glycosylated monovalent IgG4. Protein concentrations were assayed using a MSD immunoassay with capture of the antibodies using an anti-human IgG4 Fc polyclonal antibody and detection using an anti-human lambda light chain monoclonal antibody (FIG. 5). For each group WinNoLin software was used to calculate the pharmacokinetic parameters of area under the concentration-time curve from time zero extrapolated to infinity (AUCINF), clearance, beta half-life and maximum concentration (Cmax) using either non-compartmental analysis or two-compartmental modeling, the results are shown in Table 12. The half-life of the monovalent IgG4 antibodies is approximately 20 hours compared to the wild type IgG4 which has a 13 day half-life. Although the serum half-life is less than that seen for intact IgG4, a serum half-life of 20 hours for a monovalent antibody represents a significant improvement over the typical half-life of a Fab molecule in rodents, which is typically between 0.5 and 3.5 hours (see, e.g., [8], [9], [10], and [11]). The shorter serum half-life may be due to increased glomerular filtration of the smaller monovalent antibodies and/or loss of avidity for FcRn.

TABLE 12
Non-compartmental and two-compartmental analysis of
pharmacokinetic parameters for a wild type IgG4, glycosylated
monovalent IgG4 and aglycosylated monovalent IgG4.

| | | | | |
|---|---|---|---|---|
| | | Half | Half | |
| Parameter | Unit | Aglyco IgG4 | Glyco IgG4 | WT IgG4 |

Non-compartmental analysis

| | | | | |
|---|---|---|---|---|
| Half-life | Days | 0.86 | 0.86 | 13.89 |
| Cmax | ug/mL | 293.26 | 244.71 | 262.31 |
| AUCINF | Day * ug/mL | 131.83 | 178.93 | 1896.33 |
| Clearance | mL/Day/kg | 75.85 | 55.89 | 5.27 |

Two-compartmental modeling

| | | | | |
|---|---|---|---|---|
| Half-life | Days (SD) | 0.85 (0.08) | 0.87 (0.08) | 13.36 (4.12) |
| Clearance | mL/Day/kg (SD) | 119.6 (12.1) | 103.9 (11.3) | 5.32 (1.1) |

### 8.9 Example 9

### Mutagenesis of Amino Acids in the Mouse IgG1 CH3-CH3 Interface Region and Analysis by SEC-MALLS and HPLC

A number of animal model systems, including mouse models, are commonly used to evaluate the efficacy of protein-based therapeutics. These studies can rely on the use of surrogate molecules such as mouse antibodies, or fusion proteins that incorporate a mouse Fc region. An additional mutagenesis screen was performed to identify Fc mutations useful for the generation of monomeric mouse antibodies. Hingeless mouse IgG1 Fc domains with a number of site directed mutations were generated in the same manner as for the human constructs in Example 5. The choice of mutations was largely driven by the data obtained from the human monomeric Fc engineering. HPLC and SEC-MALLS was performed to determine the nature of the mutant mouse IgG1 Fc, with the data summarised in Table 13. As summarized in Table 13, the majority of mutations that lead to the formation of a monomeric human Fc domain do not lead to the formation of a monomeric mouse Fc domain. However, the mutation F405R generates a mouse IgG1 Fc domain that is predominantly monomeric, and a number of the mutations generate mouse IgG1 Fc domains that are found in monomer-dimer equilibrium.

TABLE 13
A summary of the hingeless mouse IgG1 Fc mutants analysed by size
exclusion chromatography using a Superdex 75 10/300 column at a
flow rate of 0.5 ml/min. The amino acids are numbered according to
alignment with a human CH3 domain. The samples are ordered by
retention time with calibration of the column used to estimate molecular
weight. The calculated molecular weight from multi-angle laser light
scattering is also shown for those samples that the data is available for.

| | | | SEC | MALLS |
|---|---|---|---|---|
| IgG1 mouse Fc | Analysis | RT (min) | (kDa) | (kDa) |
| WT | Dimer | 19.9 | 58.5 | 54 |
| T366R | Dimer | 20.1 | 54.0 | |
| Y349D/P354D | Dimer | 20.5 | 52.5 | |
| I351D | Dimer | 20.5 | 52.5 | |
| S364R | Dimer | 20.5 | 52.5 | |
| Q357W | Dimer | 20.5 | 52.5 | |
| S364R/K409R | Dimer | 20.6 | 51.5 | |
| F405Q | Dimer | 20.6 | 51.5 | |
| I351R | Dimer | 20.6 | 51.5 | |
| Q357R | Dimer | 20.6 | 51.5 | |
| K409R | Dimer | 20.6 | 51.5 | |
| T394R | Dimer | 20.6 | 51.5 | |
| T394D | Dimer | 20.6 | 51.5 | |
| T366W/M368W | Dimer | 20.8 | 50.0 | |
| F405Q/K409R | Dimer | 20.8 | 50.0 | |
| T394D/K409R | Dimer | 20.8 | 50.0 | 55 |
| D399R/K409R | Equilibrium | 21.1 | 47.5 | 48 |
| S364W/M368W/K409R | Equilibrium | 21.8 | 41.5 | |
| Y407R/K409R | Equilibrium | 21.9 | 41.0 | |
| S364W/M368W | Equilibrium | 22.1 | 39.5 | |
| Y407R | Equilibrium | 22.1 | 39.5 | |
| D399R | Equilibrium | 22.2 | 38.5 | 48 |
| F405R | Monomer | 22.9 | 33.5 | 30 |
| M368R | Equilibrium | 22.9 | 33.5 | 36 |
| | | (and 21.5) | | |
| F405R/K409R | Monomer | 23.1 | 32.0 | 29 |

All publications, patents and patent applications mentioned in this specification are herein incorporated by reference into the specification to the same extent as if each individual publication, patent or patent application was specifically and individually indicated to be incorporated herein by reference.
[1] Persic, L. et al. Gene. 187(1):9-18, 1997
[2] Clackson, T. and Lowman, H. B. Phage Display—A Practical Approach, 2004. Oxford University Press
[3] Krapp, S., Mimura, Y., Jefferis, R., Huber, R. & Sondermann, P. Structural analysis of human IgG-Fc glycoforms reveals a correlation between glycosylation and structural integrity. Journal of molecular biology 325, 979-989 (2003)
[4] Corper, A. L. et al. Structure of human IgM rheumatoid factor Fab bound to its autoantigen IgG Fc reveals a novel topology of antibody-antigen interaction. Nature structural biology 4, 374-381 (1997).
[5] DeLano, W. L. The PyMOL User's Manual. (DeLano Scientific, Palo Alto, Calif., USA; 2002
[6] Tsai, C. J., Lin, S. L., Wolfson, H. J. & Nussinov, R. A dataset of protein-protein interfaces generated with a sequence-order-independent comparison technique. Journal of molecular biology 260, 604-620 (1996)
[7] Dall'Acqua, W., Simon, A. L., Mulkerrin, M. G. & Carter, P. Contribution of domain interface residues to the stability of antibody CH3 domain homodimers. Biochemistry 37, 9266-9273 (1998).
[8] Chapman et al. (1999). Therapeutic antibody fragments with prolonged in vivo half-lives. Nature Biotechnology, 17, 780-783.
[9]. Nguyen et al. (2006). The pharmacokinetics of an albumin-binding Fab (AB.Fab) can be modulated as a function of affinity for albumin. Protein Engineering, Design and Selection, 19, 291-297.
[10] Pepinsky et al. (2011). Production of a PEGylated Fab′ of the anti-LINGO-1 Li33 antibody and assessment of its biochemical and functional properties in vitro and in a rat model of remyelination. Bioconjugate Chemistry, 22, 200-210.
[11] Valentine et al. (1994). Anti-phencyclidine monoclonal Fab fragments markedly alter phencyclidine pharmacokinetics in rats. The Journal of Pharmacology and Experimental Therapeutics, 269, 1079-1085.

1. A polypeptide comprising IgG immunoglobulin Fc region, wherein the Fc region comprises one or more amino acid substitutions that inhibit dimer formation of the Fc region, wherein the substitutions are at one or more of the following amino acids according to the Kabat EU numbering system: 349, 351, 354, 356, 357, 364, 366, 368, 370, 392, 394, 399, 405, 407, 409, 409 and 439.

2. The polypeptide of claim 1 further comprising a target-specific binding portion selected from the group consisting of:
(i) an immunoglobulin light chain variable region and an immunoglobulin heavy chain variable region that associate to form the target-specific binding portion;
(ii) a domain antibody (dAb); and
(iii) a protein scaffold.

3. (canceled)

4. The polypeptide of claim 1, wherein said polypeptide is a fusion protein comprising an immunoglobulin Fc region fused to a therapeutic polypeptide.

5-7. (canceled)

8. The polypeptide of claim 1, wherein the one or more amino acids are substituted with an amino acid selected from the group consisting of:
(i) an amino acid having a positively charged side chain;
(ii) an amino acid having a negatively charged side chain;
(iii) an amino acid having a hydrophilic side chain; and
(iv) an amino acid having a large side chain.

9. (canceled)

10. The polypeptide according to claim 1, wherein the Fc region is from a human IgG immunoglobulin or a mouse IgG immunoglobulin.

11. (canceled)

12. The polypeptide according to claim 10, wherein the Fc region is from an IgG1, IgG2, IgG3 or IgG4 immunoglobulin.

13. (canceled)

14. The polypeptide according to claim 1, wherein one or more of the following amino acid positions have been substituted with an amino acid having a positively charged side chain: 351, 356, 357, 364, 366, 368, 394, 399, 405 and 407, wherein the amino acid having a positively charged side chain is selected from: Arginine, Histidine and Lysine.

15. The polypeptide according to claim 1, wherein one or more of the following amino acid positions have been substituted with an amino acid having a negatively charged side chain: 349, 351, 394, 407 and 439, wherein the amino acid having a negatively charged side chain is selected from: Aspartic acid and Glutamic acid.

16. The polypeptide according to claim 1, wherein one or more of the following amino acid positions have been substituted with an amino acid having a large side chain: 357, 364, 366, 368, and 409, wherein the amino acid having a large side chain is selected from: Tryptophan, Phenylalanine and Tyrosine.

17. The polypeptide according to claim 1, wherein one or more of the following amino acid positions have been substituted with an amino acid having a hydrophilic side chain: 366, 405 and 407, wherein the amino acid having a hydrophilic side chain is selected from: Glutamine, Asparagine, Serine and Threonine.

18-28. (canceled)

29. The polypeptide of claim 1, wherein the Fc region comprises one or more of the following amino acid substitutions: L351R, L351D, E357R, E357W, S364R, T366R, L368R, T394R, T394D, D399R, F405R, F405Q, Y407R, Y407D, K409W and R409W.

30. The polypeptide of claim 1, wherein the Fc region comprises at least two amino acid substitutions that inhibit dimer formation.

31. (canceled)

32. The polypeptide of claim 30, wherein the amino acid substitutions are selected from the group consisting of: Y349D, L351D, L351R, S354D, E356R, D356R, S364R, S364W, T366Q, T366R, T366W, L368R, L368W, T394D, T394R, D399R, F405A, F405Q, Y407A, Y407Q, Y407R, K409R, and K439D.

33. The polypeptide of claim 30, wherein the Fc region comprises one or more of the following sets of amino acid substitutions: Y349D/S354D, L351D/T394D, L351D/K409R, L351R/T394R, E356R/D399R, D356R/D399R, S364R/L368R, S364W/L368W, S364W/K409R, T366R/Y407R, T366W/L368W, L368R/K409R, T394D/K409R, D399R/K409R, D399R/K439D, F405A/Y407A, F405Q/Y407Q and T366Q/F405Q/Y407Q.

34. The polypeptide according to claim 1, wherein said polypeptide comprises an immunoglobulin heavy chain having a deleted or mutated hinge region.

35-40. (canceled)

41. The polypeptide according to claim 1, wherein at least 70% of the polypeptide present in a solution is monomeric as determined by SEC-MALLS or AUC.

42-43. (canceled)

44. A nucleic acid molecule encoding a polypeptide according to claim 1.

45. A host cell transformed with a nucleic acid molecule according to claim 44.

46. (canceled)

47. A pharmaceutical composition comprising the polypeptide of claim 1 and a pharmaceutically acceptable excipient.

48-49. (canceled)

50. The pharmaceutical composition of claim 47 for use as a medicament.
